(19) United States
(12) Patent Application Publication
(10) Pub. No.: US 2017/0128479 A1
(43) Pub. Date: May 11, 2017

(54) TREATMENT OF PEROXISOME BIOGENESIS DISORDER

(71) Applicant: The Hospital for Sick Children, Toronto (CA)

(72) Inventor: Peter K. KIM, North York (CA)

(21) Appl. No.: 15/319,209

(22) PCT Filed: Nov. 14, 2014
(86) PCT No.: PCT/CA2014/000816

§ 371 Date: Dec. 15, 2016

Related U.S. Application Data
(60) Provisional application No. 62/014,376, filed on Jun. 19, 2014.

(51) Int. Cl. A61K 31/7048; A61K 31/4706; A61K 31/382; A61K 31/409; A61K 31/198; A61K 31/55; A61K 31/675; A61K 31/5377

(52) U.S. Cl. CPC A61K 31/7048; A61K 31/675; A61K 31/4706; A61K 31/5377; A61K 31/409; A61K 31/198; A61K 31/55; A61K 31/382

(57) ABSTRACT

A method is described for treating a peroxisome biogenesis disorder by administering to an individual in need thereof an effective amount of an autophagy inhibitor. Uses, compositions, and commercial packages are also described. The peroxisome biogenesis disorder may be Zellweger syndrome, neonatal adrenoleukodystrophy, Refsum disease, or cerebrohepatorenal syndrome. The autophagy inhibitor may be chloroquine diphosphate; hydroxychloroquine sulfate; verteporfin; difluoromethylornithine; clarithromycin; clomipramine; desmethylclomipramine hydrochloride, anisomycin; Spautin-1; U0126; SP600125; Wortmannin; LY294002; Bafilomycin; Forskolin; Melatonin; 1-((2-(diethylamino)ethyl)amino)-4-methylthioxanthen-9-one; 1-(2-diethylaminoethylamino)-4-(hydroxymethyl)-9-thioxanthenone; or N-[[1-[[2-(diethylamino)ethyl]amino]-9-oxo-9H-thiaxanthen-4-yl]methyl]methanesulfonamide.

### CROSS REFERENCE TO RELATED APPLICATIONS

This application claims the benefit of priority of U.S. Provisional Patent Application No. 62/014,376 filed Jun. 19, 2014, which is hereby incorporated by reference in its entirety.

### FIELD

The present disclosure relates to methods, compositions, and uses thereof for treatment of a peroxisome biogenesis disorder.

### BACKGROUND

Peroxisomes are vital metabolic organelles that are expressed in all eukaryotic cells except erythrocytes. They are highly pleomorphic and dynamic organelles that change in shape and numbers based on the specific metabolic needs of different tissues and cell types. Peroxisomes are best known for their catabolism of very long chain fatty acids (VLCFA: carbon chain >22), as well as their antioxidant activity (see Singh et al., 2004; and Uto et al., 2009). Peroxisomes are also involved in the synthesis of lipids such as bile acids, docosohexanoic acid, an omega 3 fatty acid, and plasmalogens, a specialized class of membrane phospholipids. Although a single cell may be able to survive without peroxisomes in certain conditions, this organelle is essential for the proper function and viability of tissues and organs. The importance of peroxisomes is most evident by the group of diseases resulting from the lack of functioning peroxisomes called Peroxisome Biogenesis Disorders or “PBD” (Weller et al., 2003).

Peroxisome Biogenesis Disorders are a continuum of disorders that divide into three phenotypes based on the severity of the disorder. Zellweger Syndrome (ZS) is the most severe form; followed by neonatal adrenoleukodystrophy (NALD), an intermediate form and infantile Refsum disease (IRD), which is the least severe. Children with ZS present early in the neonatal period with profound hypotonia, facial dysmorphisms, liver dysfunction, seizures, and rarely survive past their first year of life. At a biochemical level, laboratory tests show profound deficiencies in peroxisomal metabolism and cells from these patients show near absence of functional cellular peroxisomes. NALD and IRD are more difficult to distinguish from each other, form the majority of cases, and can live through adulthood. Both may present soon after birth, but are often diagnosed later by developmental delays, mild liver dysfunction, and hearing and visual impairment leading to their classification as deaf-blind children. A proportion of NALD-IRD children will develop a leukodystrophy that represents destruction of normal myelin. They may also develop adrenal gland insufficiency and osteopenia, the latter causing pathological fractures. Cells from patients with NALD-IRD contain more functional peroxisomes than ZS patients, and laboratory tests show higher residual peroxisome metabolism. Clinical features result from secondary to deficiency of products made by the peroxisome (e.g plasmalogens) and toxicity of substrates that accumulate (such as very long chain fatty acids).

These autosomal recessive diseases are derived from dysfunction of peroxisomes and current treatment is primarily focused on supportive care, dietary management, symptomatic therapy and treatment strategies involving the use of pharmacological induction of peroxisomes. To date, therapeutic interventions for PBDs have targeted biochemical defects of peroxisomal function in patients. While this approach appears to have some effect on the milder forms of PBDs, it can be highly variable in PBD patients and not effective in patients with Zellweger syndrome. These disorders are thought to be disorders of peroxisome biogenesis per se, and thus have been grouped together under the name: peroxisome biogenesis disorders.

Genetically, PBD is caused by mutations in one of 13 PEX genes that are required for peroxisome assembly. However, the greatest number of mutations occur in one of the three genes whose proteins constitute the peroxisomal AAA ATPase complex: PEX1, PEX6, and PEX26. The term “AAA ATPase” is an abbreviation for ATPases Associated with diverse cellular Activities. Mutations in these 3 PEX genes make up 90% of all reported cases of PBD, with mutations in PEX1 accounting for more than 65% of all reported cases of PBD. The two most common PEX1 mutations are the PEX1 null allele, PEX1-p.Ile700fs, and a missense allele, PEX1-p.Gly843Asp (PEX1-G843D), that encodes a misfolded protein with residual functions. The homozygous PEX1-p.Ile700fs mutation is associated with the severe form of PBD, whereas the presence of at least one PEX1-G843D ensures an intermediate or milder phenotype. It is important to note that the intermediate and milder phenotypes cause a progressive loss of clinical functions over time and thus, would benefit from therapeutic interventions that can restore peroxisomal function.

Peroxisome biogenesis disorders clinically differ from single peroxisomal enzyme mutational peroxisomal diseases such as X-linked Adrenoleukodystrophy (XALD). X-linked Adrenoleukodystrophy results from the defect in the ABCD transporter required for very long chain fatty acid import into peroxisomes and results in the loss of myelin sheath in the white brain matter (leukodystrophy). Although leukodystrophy is a phenotype found some PBD patients, it is not present in all patients. Instead, PBD effects many more tissues and organelles than XALD.

The role of the peroxisomal AAA ATPase complex in peroxisome biogenesis is to remove the peroxisomal matrix (lumen) protein receptor, PEX5, from the peroxisomal membrane (Platta et al., 2005). The peroxisome has a unique mechanism of importing its matrix proteins directly from the cytosol into peroxisomes by the use of a transient pore formed by PEX5 and two peroxisomal membrane proteins, PEX14 and PEX13 (Thorns et al., 2006).

FIG. 1 illustrates that to disassemble the transient pore, PEX5 is selectively modified by a ubiquitin tag (a 76 residue protein) and removed from the lipid bilayer by the peroxisomal AAA ATPase complex. More specifically, FIG. 1 shows a matrix protein import pathway. Peroxisome matrix protein (small, unlabeled circle) is bound by the cytosol receptor PEX5. PEX escorts the peroxisome matrix protein to the peroxisomal membrane by binding to the peroxisomal importer complex (PEX14 and PEX13) where it forms a transient pore. The cargo is released into the matrix (lumen) of peroxisomes. The transient pore is disassembled by the removal of PEX5. PEX5 is removed by its ubiquitination by the peroxisomal Ubiquitin E3 ligases (not depicted). Ubiquitinated PEX5 is then removed from the lipid bilayer by the exporter consisting of the AAA ATPase complex PEX1, PEX6 and PEX26. The released PEX5 is deubiquitinated and recycled to repeat the import of other matrix proteins. The absence of the AAA ATPase function does not prevent the import of matrix proteins, but rather it is thought that its absence limits the available cytosolic PEX5 to import newly synthesized matrix proteins (see Ma et al., 2009).

Superfluous and damaged peroxisomes are removed from the cell by a degradation process called macroautophagy (herein referred as autophagy) as described in FIG. 2. Autophagy is a conserved cellular process wherein a double membrane phagophore (autophagosomes) forms to sequester large cytosolic components to be delivered to lysosomes for degradation. Damaged or excess peroxisomes are selectively targeted to lysosomes by autophagosomes. In mammalian cells, a peroxisome is designated for degradation by the accumulation of ubiquitinated protein on the peroxisomal membrane (see Kim et all, 2008). Ubiquitinated peroxisomes then recruit autophagy adaptor proteins, NBR1 and p62, which target the marked peroxisomes to nascent autophagosomes to be sequestered for degradation (Kim et al, 2008; Deosaran et al., 2013).

FIG. 2 is a schematic representation of the degradation of peroxisomes by autophagy pathway. Peroxisome degradation is initiated the emergence of two pathways. The first pathway is the formation of autophagosomes (A1), which are the initial steps of the macroautophagy pathway (herein referred to as autophagy). Autophagy starts with the formation of a double membrane structure called autophagosomes which sequesters the cytoplasma. The second pathway is the designation of a peroxisome for degradation (A2). Here the peroxisomes become ubiquitinated (ub) by an unknown mechanism to ‘mark’ it for degradation. B. Targeting. Here the two pathways emerge. Autophagy adaptor proteins NBR1 and p62 mediate the targeting of ubiquitinated peroxisome to autophagosomes by bind to the ubiquitinated substrates on peroxisomes, and bind to the autophagy factor (LC3) on the nascent autophagosome C. Degradation. The nascent autophagosome fuses with itself enclosing its contents to form the autophagosome. The autophagosome then fuses with the lysosome to form the autolysosome. Lysosomal hydrolytic enzymes degrade the ubiquitinated contents and the inner membrane of the autophagosome. Also shown are the sites of action of four different autophagy inhibitors. Vertephorfin and DFMO act at the early stages of autophagy and prevent the formation of autophagosomes. Chloroquine and FTY720 act to prevent the acidification of lysosomes.

Autophagy inhibitors are known, and some are discussed below.

U.S. Pat. No. 8,524,762 entitled “Thioxanthone-based autophagy inhibitor therapies to treat cancer”, describes thioxanthone-based autophagy inhibitor therapies to treat cancer. Some forms of cancer require the upregulation of autophagy for survival thereby inhibiting autophagy which will make cancer cells more susceptible to chemotherapy and radiotherapy or other cancer treatments. Presently there are various clinical studies using Chloroquine along with cancer chemotherapy drugs to treat cancer.

U.S. Pat. No. 8,440,695 teaches the use of chloroquine to treat metabolic syndrome.

U.S. Pat. No. 8,466,311 entitled “Neuroprotective compositions and methods” describes the use of Nrf2 activators to enhance the upregulation of antioxidant defense within the cell, with the intention of down-regulating oxidative stress for neuroprotection.

U.S. Pat. No. 7,994,222 entitled “Monitoring of the inhibition of fatty acid synthesis by iodo-nitrobenzamide compounds” describes the use of iodo-nitrobenzamide compounds and metabolites to inhibit the activation or increase in fatty acid metabolism for the treatment of cancer.

Different approaches have been taken to investigate and address peroxisome biogenesis disorders. Wei et al. (2000) used the peroxisome proliferator, 4-phenylbutyrate, to show that increasing peroxisome numbers in PBD cells from patients with the intermediate and milder forms could reduce very long chain fatty acid levels. Examples from within the patent literature include U.S. Pat. No. 6,197,543 entitled “Human vesicle membrane protein-like proteins” which describes nucleic acid and amino acid sequences of three human vesicle membrane protein-like proteins and to the use of these sequences in vesicle trafficking disorders. U.S. Pat. No. 8,206,947 entitled “Human transmembrane proteins” describes nucleic acid and amino acid sequences of human transmembrane proteins for treating or preventing a disorder associated with decreased expression or activity of the transmembrane proteins. Further, U.S. Pat. No. 8,343,996 entitled “Azaquinolinone derivatives and uses thereof.” describes treatment of certain disorders caused by abnormal folding or aggregation of proteins. However, there is still an unmet need to address peroxisome biogenesis disorders, including the three major subgroupings, Zellweger syndrome spectrum, neonatal adrenoleukodystrophy and infantile Refsum disease.

There is no cure for PBDs, which are currently treated with symptomatic therapy. There is, therefore, a need for strategies to address treatment of peroxisome biogenesis disorders.

### SUMMARY

It is an object of the present disclosure to obviate or mitigate at least one disadvantage of previous methods for treating peroxisome biogenesis disorders.

There is provided herein a method of treating a peroxisome biogenesis disorder by administering to an individual in need thereof an effective amount of one or more autophagy inhibitor. Further, there is described herein the use of an autophagy inhibitor (or more than one inhibitor) for treating a peroxisome biogenesis disorder in an individual in need thereof, or for preparation of a medicament for such treatment. A composition is provided herein, for use in treating a peroxisome biogenesis disorder in an individual in need thereof comprising one or more autophagy inhibitor and an acceptable diluent. Further, a commercial package is provided for treating a peroxisome biogenesis disorder comprising one or more autophagy inhibitor and instructions for use.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 shows a known matrix protein import pathway.

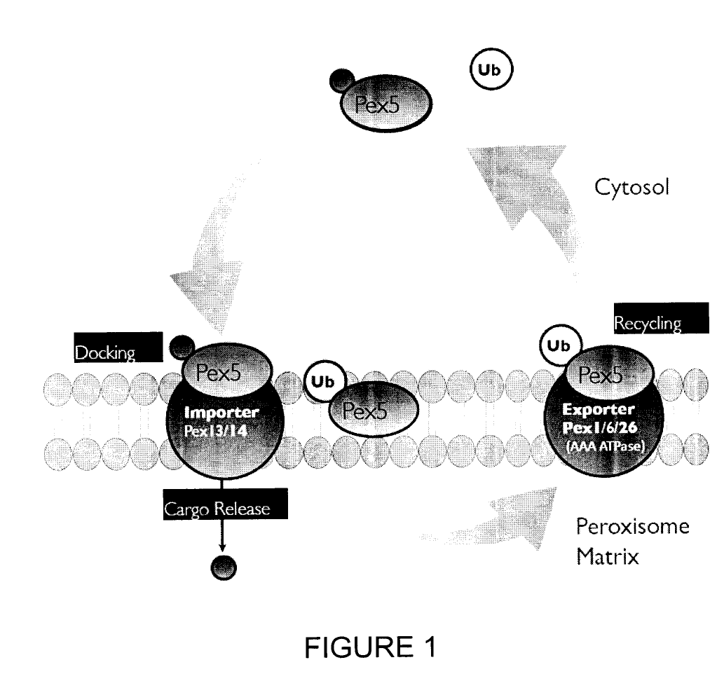

FIG. 2 is a schematic representation of the degradation of peroxisomes by the autophagy pathway.

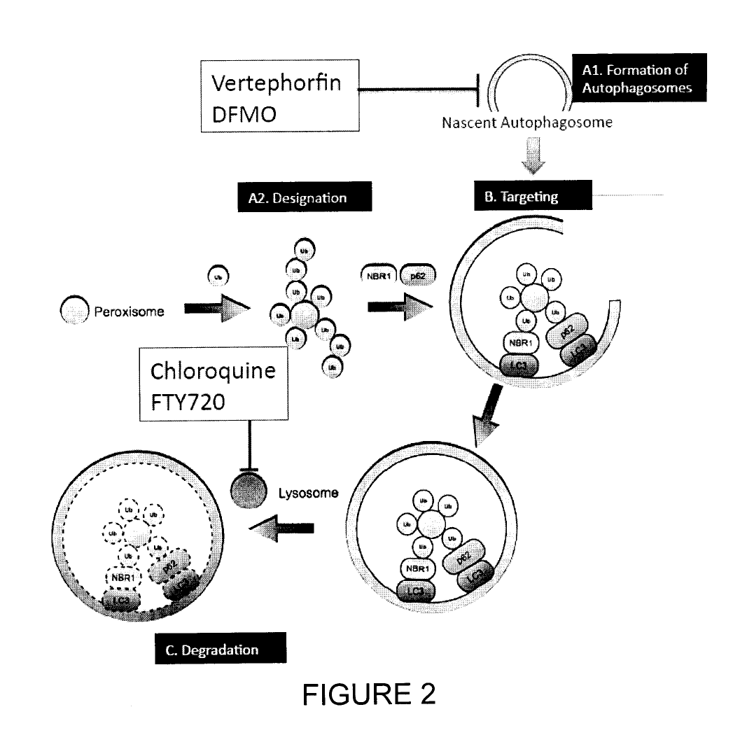

FIG. 3 provides a model of AAA ATPase dysfunction in PBD.

FIG. 4 shows that disruption of the AAA ATPase complex causes a decline in peroxisome numbers.

FIG. 5 shows data illustrating that disruption of autophagy rescues AAA ATPase mediated peroxisome loss.

FIG. 6 shows data illustrating that autophagy inhibitors recruit peroxisome number.

FIG. 7 provides data to show that inhibiting autophagy in PBD patient cells restores peroxisome numbers.

FIG. 8 shows that inhibiting autophagy restores peroxisome function in PBD cells.

FIG. 9 shows data for HeLa cells transfected with siRNA (Panel A), a graph of PMP70 intensity per unit volume (Panel B); and cell lysate immunoblotting with PMP70 and GAPDH antibodies in (Panel C); illustrating that depletion of the AAA ATPase complex reduces peroxisome number.

FIG. 10 shows Wild-type and Atg5−/− MEFs transfected with siRNA against PEX26 (Panel A), and the total PMP70 intensity per volume of each cell (Panel B).

FIG. 11 shows HeLa cells transfected with non-targeting siRNA (siCTRL), siRNA against PEX1, siRNA against PEX26, all +/−siRNA against ATG12 and immunostained for PMP70 (Panel C); and a graph of the number of PMP70 puncta per volume of each cell (Panel D)

FIG. 12 shows HeLa cells transfected with non-targeting siRNA (siCTRL), siRNA against PEX1, siRNA against PEX26, all +/−siRNA against NBR1 (Panel E); and PMP70 puncta fluorescence per volume of each cell (Panel F).

FIG. 13 shows HeLa cells were transfected with GFP-LC3 and non-targeting siRNA (siCTRL), siRNA against PEX1, siRNA against PEX14, siRNA against PEX26, all +/−siRNA against NBR1 (Panel A); a gGraph of the number of GFP-LC3 puncta per volume of each cell (um³) normalized to siCTRL (Panel B); and an immunoblot of total cell lysates prepared from HeLa cells treated with non-targeting siRNA, or siRNA against PEX1 or PEX26 as well as with E-64 and leupeptin inhibitors, immunoblotted with LC3 and GAPDH to confirm LC3-II increase seen (Panel C).

FIG. 14 shows HeLa cells transfected with non-targeting siRNA (siCTRL), siRNA against PEX1, siRNA against PEX14, siRNA against PEX26, all +/−siRNA against NBR1 (Panel D); and a graph of the Oxyburst fluorescence intensity normalized to control of at least 30 cells per trial (Panel E).

FIG. 15 shows total cell lysates prepared from cells transfected with GFP-Ub and RFP-SKL in a 1:4 ratio, and with non-targeting siRNA (siCTRL), or siRNA against PEX14, PEX1, or PEX26, and immunoblotted with Pex5 (Panel F); and immunoprecipitation of HA (HA-Ub) from HEK293 cells expressing HA-Ub under the control of a tamoxifen-inducible promoter that have been treated with siRNA against non-targeting siRNA, or siRNA against PEX14, PEX1, or PEX26 (Paned G).

FIG. 16 shows Control, PEX1-G843D homozygous, and PEX1-null fibroblasts treated with Bafilomycin (C₂=2 nM), Chloroquine (C₂=20 μM), LY294002 (C₂=5 mM), immunostained for PMP70. Scale bars, 20 μM (Panel A); and a graph of the number of PMP70 puncta per volume of each cell (Panel B).

FIG. 17 shows total cell lysates were prepared from fibroblasts treated with the various drugs for 24- and 48-hours and immunoblotted with Pex14 and GAPDH (Panel C); and PEX1-G843D homozygous fibroblasts stably expressing GFP-PTS1 (PEX1-G843D-PTS1) treated with various drugs (Panel D).

FIG. 18 shows a graph of the number of GFP-PTS1 puncta per volume of cell (1×10⁻³ μM) of at least 30 cells per trial for different treatments.

FIG. 19 shows viabilitiy of control fibroblasts treated with varying concentrations of chloroquine (0, 1, 5, 10, 20, 50, 100 μM) for 24-, 48-, 72-, and 96-hours (Panel A); and PEX1-G843D-PTS1 cells either not treated, or treated with 5 or 10 μM Chloroquine for 48- or 72-hours, and fixed (Panel B)

FIG. 20 shows a graph of the number of GFP-PTS1 puncta per volume of each cell (Panel C), PEX1-G843D-PTS1 fibroblasts either not-treated, or treated with 5 μM Chloroquine for 48- or 72-hours as indicated and fixed and immunostained for PMP70 (Panel D), a graph of the number of PMP70 puncta per volume of each cell (Panel E).

FIG. 21 shows total cell lysates were prepared from control, PEX1-, and PEX1-G843D-fibroblasts treated with 5 or 10 μM Chloroquine (Panel F); and a graph of functional recovery in PEX1-G843D homozygous and hemizygous primary fibroblasts cultured in regular media or regular media+chloroquine for 144-hours (Panel G).

FIG. 22 shows images of PEX1-G843D-PTS1 fibroblasts treated with 5 μM Chloroquine on days 0 (control), 3, 6, 9, 12, and 15.

FIG. 23 shows (Panel B) a graph of the number of GFP-PTS1 puncta per volume of each cell (um³) normalized to the non-treated fibroblasts (Panel B); and a graph of the % viability relative to not-treated cells (Panel C).

### DETAILED DESCRIPTION

Generally, the present disclosure provides the use of autophagy inhibitors for the treatment of peroxisome biogenesis disorders. Modulation of autophagy activity of peroxisome degradation offers a viable treatment option to increase the number and normal functioning of peroxisomes, thereby preventing further loss of peroxisome numbers.

Methods, uses, compositions and commercial packages are described herein pertaining to the use of autophagy inhibitors for the treatment of peroxisome biogenesis disorders.

It has surprisingly been found that PBDs can be treated by addressing the increased rate of peroxisome degradation or autophagy with autophagy inhibition. For both those patients with severe forms who have greatly limited life spans, as well as those with milder form of PBDs who live to the second decade of life, treatments such as those described herein can improve their quality of life.

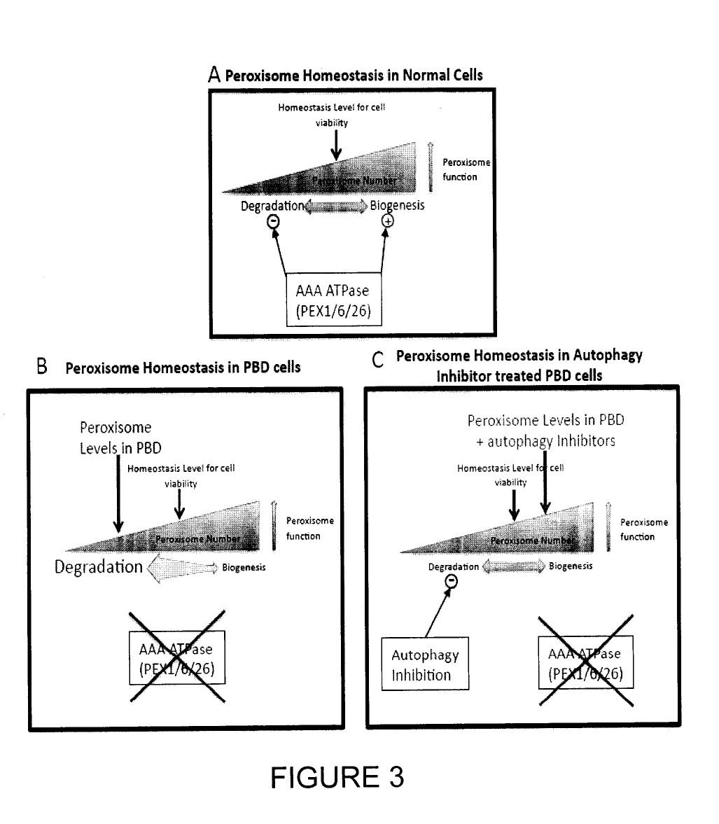

As described herein, it has now been shown that the AAA ATPase is also involved in the peroxisome quality control process by preventing the degradation of functional peroxisomes. This was heretofore unrecognized. FIG. 3 provides a model of AAA ATPase dysfunction in PBD. Peroxisomal AAA ATPase includes PEX1, PEX6 and PEX26. Its defect is responsible for about 90% of all reported PBD cases. Panel (A) illustrates that in normal cells, AAA ATPase maintains peroxisome level by removing ubiquitinated membrane proteins from peroxisomes. This promotes biogenesis and prevents degradation of peroxisomes. Peroxisomes with insufficient AAA ATPase function are degraded. Panel (B) illustrates that in PBD resulting from lack of AAA ATPase function, newly formed peroxisomes are rapidly degraded due to the accumulation of ubiquitinated membrane proteins. This results in a lower peroxisome levels. Panel (C) shows that peroxisomes are mainly degraded by the autophagy pathway. Peroxisome loss can be repressed by inhibiting autophagy. Therefore by inhibiting autophagy peroxisome function can be restored by an increase the number of peroxisomes in the cell. What has not been well understood previously, is how superfluous and damaged peroxisomes are selectively ubiquitinated for degradation.

Inhibiting peroxisome degradation by inhibiting autophagy can be undertaken using autophagy inhibitors, such as those described herein, to increase functional peroxisomes in cell lines with defect in AAA ATPase defect. The treatment of peroxisome biogenesis disorders in this manner addresses an unmet need for patients. Peroxisome biogenesis disorders are rare, and thus benefit from treatment with orphan drugs. Utilization of known compounds, some of which are FDA approved, as autophagy inhibitors can reduce the time frame required to make treatments available so as to treat or address some of the common phenotypes of the most prevalent forms of peroxisome biogenesis disorders.

As used herein, “treatment” refers to addressing a disorder an individual is already experiencing symptoms of and tendencies toward, in a manner that results in improvement or a lack of further decline, where decline is expected. Treatment need not entirely cure or eradicate the disorder or the symptoms thereof, but may simply lessen the strength or duration one or more of the symptoms experienced. Improvement may be determined by the individual, by a health care professional, or may be determined using accepted parameters as may be assessed by a clinician or other care giver. Amelioration of a symptom without entirely eradicating the symptom is considered as treatment.

There is provided herein a method of treating a peroxisome biogenesis disorder by administering to an individual in need thereof an effective amount of an autophagy inhibitor. The peroxisome biogenesis disorder may be Zellweger syndrome, neonatal adrenoleukodystrophy, Refsum disease, cerebrohepatorenal syndrome, or a combination of these. The autophagy inhibitor may comprise chloroquine diphosphate; hydroxychloroquine sulfate; verteporfin; difluoromethylornithine; clarithromycin; clomipramine; desmethylclomipramine hydrochloride, anisomycin; Spautin-1; U0126; SP600125; Wortmannin; LY294002; Bafilomycin; Forskolin; or Melatonin. Exemplary compounds are: LY294002, Bafilomycin, and chloroquine.

Thioxanthone-based autophagy inhibitors may be used, for example as described in U.S. Pat. No. 8,524,762, the entirety of which is herein incorporated by reference. While this document teaches that these inhibitors may have an impact on cancer, they can also be used as one or more of the autophagy inhibitors described in the instant methods and compositions. For example, thioxanthone-based inhibitors such as 1-((2-(diethylamino)ethyl)amino)-4-methylthioxanthen-9-one, 1-(2-diethylaminoethylamino)-4-(hydroxymethyl)-9-thioxanthenone, or N-[[1-[[2-(diethylamino) ethyl]amino]-9-oxo-9H-thiaxanthen-4-yl] methyl] methanesulfonamide, may be used, or indazole analogues thereof, or salts thereof.

The use of such an autophagy inhibitor is also described for treating one or more of the peroxisome biogenesis disorders in an individual in need thereof, as well as the use of the autophagy inhibitor for preparation of a medicament for use in treating a peroxisome biogenesis disorder in an individual in need thereof.

There is also described herein a composition for use in treating a peroxisome biogenesis disorder in an individual in need thereof comprising an autophagy inhibitor and an acceptable diluent.

Further, a commercial package for treating a peroxisome biogenesis disorder comprising an autophagy inhibitor together with instructions for use.

Dosage levels of the compound or compounds to be used will be considered with regard to efficacy, toxicity, etc. For those compound already having FDA approval, doses for use herein are expected to cover the ranges of dose levels already approved, and may also fall outside of these doses if higher or lower levels are found to have adequate safety and efficacy. Further, when more than one autophagy inhibitor is used simultaneously, whether in parallel co-administration, or in a combination strategy that involves staggered timing, the amounts of each inhibitor is to be considered in the context of the overall dosing strategy as a whole. When used in combination, some of the inhibitors may advantageously exhibit an additive effect or a synergy that may permit use of individual inhibitors at dosage levels that may be considered sub-clinical, were these compounds to be used alone. The additive effect in preventing peroxisome degradation, and reduction in possible side effects of each drug may be realized. It is understood that high levels of the autophagy inhibitor compounds would not be administered if efficacy in autophagy inhibition has an unacceptable toxicity or impact on a subject in terms of side effects.

Compounds

FDA-Approved compounds which show autophagy inhibition activity include but are not limited to: chloroquine diphosphate and derivatives thereof (such as hydroxychloroquine sulfate); verteporfin; difluoromethylornithine (elfornithine); clarithromycin; clomipramine, a tricyclic antidepressant, and derivatives thereof such as desmethylclomipramine hydrochloride. Natural products which show autophagy inhibition activity include forskolin, and melatonin (see for example Zheng et al., 2014). Other compounds which exhibit autophagy inhibition include anisomycin (flagecidin), an antibiotic.

Table 1 shows examples of potential autophagy inhibitors for use as described herein, which are FDA approved for unrelated indications.

TABLE 1
Autophagy Inhibitors - Examples of FDA Approved Drugs

| DRUG | Activity | FDA approved use |
|---|---|---|
| Chloroquine | Prevents lysosome acidification | Malaria |
| Verteporfin | Prevent Autophagosome | Photosensitizer for |
| | formation | photodynamic therapy to |
| | | eliminate the abnormal blood |
| | | vessels in the eye and |
| | | treatment of central serous |
| | | retinopathy |
| Clomipramine | Block autophagy flux | a tricyclic antidepressant |
| Difluoromethylornithine | Prevents Starvation induced | African trypanosomiasis |
| | Autophagy by inhibiting | (sleeping sickness) |
| | Ornithine decarboxylase. | |
| Anisomycin | P38 activator | Antibiotic |

Table 2 shows exemplary autophagy inhibitors for use as described herein, which are not FDA approved.

TABLE 2
Non-FDA Approved Compounds and/or Natural Products
for use in Autophagy Inhibition

| Name | Activity |
|---|---|
| Spautin-1 | Vps34 inhibitor |
| U0126 | ERK inhibitor |
| SP600125 | JNK inhibitor |
| Wortmannin | PI3Kinase inhibitor |
| Ly294002 | PI3Kinase inhibitor |
| Bafilomycin A1 | Prevents lysosome acidification |
| Forskolin | |
| Melatonin | |

Other compounds not shown in Table 1 or Table 2, which have an autophagy inhibition effect may also be used, such as, for example: 1-((2-(diethylamino)ethyl)amino)-4-methylthioxanthen-9-one, 1-(2-diethylaminoethylamino)-4-(hydroxymethyl)-9-thioxanthenone, or N-[[1-[[2-(diethylamino)ethyl]amino]-9-oxo-9H-thiaxanthen-4-yl]methyl]methanesulfonamide, or indazole analogues thereof, or salts thereof.

### EXAMPLES

### Example 1

### Chloroquine Diphosphate

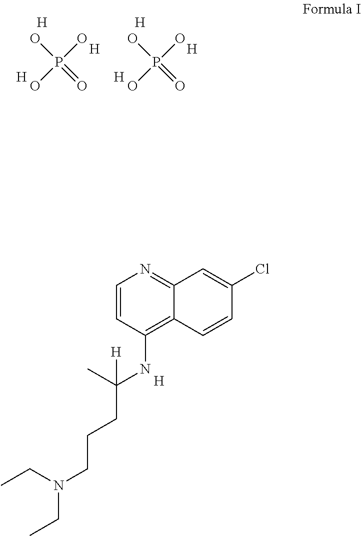

Chloroquine diphosphate is shown above as Formula I. The compound may also be referred to as Aralen phosphate, Alermine, Chingaminum, Chloroquine bis(phosphate), Aralen diphosphate, 1,4-Pentanediamine, N4-(7-chloro-4-quinolinyl)-N1,N1-diethyl-, phosphate (1:2); or 7-Chloro-4-((4-(diethylamino)-1-methylbutyl)amino)quinoline phosphate (1:2). An early description of the compound of Formula I can be found in U.S. Pat. No. 4,151,281 issued Apr. 24, 1979 entitled: Medicinal preparation for the treatment of collagenoses of a rheumatoid nature.

### Example 2

### Hydroxychloroquine Sulfate

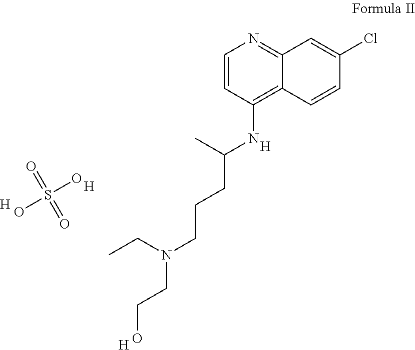

Hydroxychloroquine sulfate is shown in Formula II, and may also be referred to as rcoquin; Plaquinol; Toremonil; 2-((4-((7-Chloroquinolin-4-yl)amino)pentyl)(ethyl)amino) ethanol sulfate; 7-Chloro-4-[4-[ethyl-(2-hydroxyethyl)amino]-1-methylbutylamino]quinolone. An earlier report of this compound and its uses can be found in U.S. Pat. No. 4,151,281.

### Example 3

### Verteporfin (Visudyne)

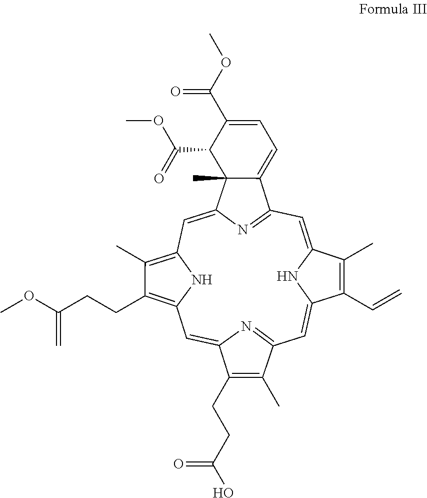 [SMILES: C=Cc1c(C)c2cc3nc(cc4[nH]c(cc5nc(cc1[nH]2)C(C)=C5CCC(=O)O)c(CCC(=C)OC)c4C)[C@@]1(C)C3=CC=C(C(=O)OC)[C@H]1C(=O)OC]

The structure of Verteporfin (Visudyne) is shown in Formula Ill. An earlier patent reporting the structure and use of this compound can be found, for example, in U.S. Pat. No. 5,707,608.

### Example 4

### Difluoromethylornithine (Elfornithine)

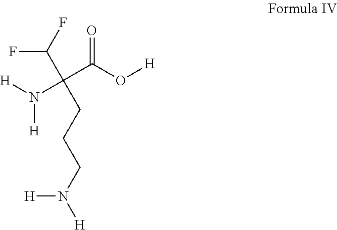 [SMILES: NCCCC(N)(C(=O)O)C(F)F]

The structure of Difluoromethylornithine (Elfornithine) is shown in Formula IV. The compound may also be referred to as ornidyl, dfmo, 2-(Difluoromethyl)ornithine, Elfornithine, 2,5-diamino-2-(difluoromethyl)pentanoic acid. An earlier report of this compound can be found in U.S. Pat. No. 4,309,442 issued Jan. 5, 1982 entitled: Method for controlling fertility in mammals.

### Example 5

### Clarithromycin (Biaxin)

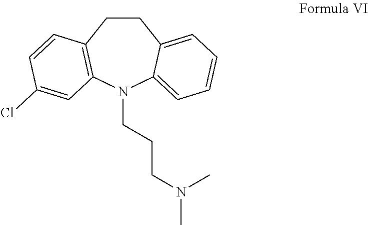 [SMILES: CN(C)CCCN1c2ccccc2CCc2ccc(Cl)cc21]

The structure of Clarithromycin (Biaxin) is shown in Formula V. The compound may also be known as Clarithromycine, 6-O-Methylerythromycin, Klaricid, Macladin, Biaxin XL, Clathromycin. An earlier report of this compound and its uses can be found, for example, in International (PCT) Patent Publication WO/1990/014094 A1, published Nov. 29, 1990 entitled: Injectable Clarithromycin Composition.

### Example 6

### Clomipramine

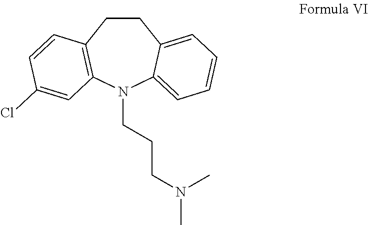 [SMILES: CN(C)CCCN1c2ccccc2CCc2ccc(Cl)cc21]

The structure of clomipramine is shown in Formula VI. The compound may also be known as Chlorimipramine, Hydiphen, Monochlorimipramine, 3-Chloroimipramine, Anafranil base, Anafranil (free base), 303-49-1, Chlomipramine; 3-(2-chloro-5,6-dihydrobenzo[b][1]benzazepin-11-yl)-N,N-dimethylpropan-1-amine. An earlier report of this compound and its uses can be found in U.S. Pat. No. 4,426,387 which issued on Jan. 17, 1984 entitled: Piperid-4-yl ureas and thio ureas used as anti-depressant agents.

### Example 7

### Desmethylclomipramine Hydrochloride

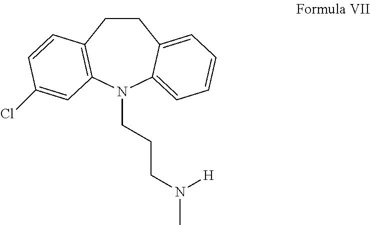 [SMILES: CNCCCN1c2ccccc2CCc2ccc(Cl)cc21]

The structure of desmethylclomipramine hydrochloride is shown in Formula VII. The compound may also be known as Demethylchlorimipramine, N-Demethylclomipramine, Desmethylchlorimipramine, Norclomipramine, 3-(2-chloro-5,6-dihydrobenzo[b][1]benzazepin-11-yl)-N-methylpropan-1-amine. An earlier report of this compound can be found, for example, in U.S. Pat. No. 4,249,002 issued on Feb. 3, 1981 entitled: Polycyclic amines and intermediates therefor.

### Example 8

### siRNA Disruption of AAA ATPASE Complex

The accumulation of ubiquitin on peroxisome membrane can signal for its degradation, and peroxisomal AAA ATPase removes ubiquitinated PEX5 from peroxisomes. In this example, the peroxisomal AAA ATPase was assessed for involvement in in peroxisome quality control. It was found that AAA ATPase prevents the accumulation of ubiquitinated PEX5 proteins by quickly removing them from the peroxisomal membrane. Thus, inhibiting the function of the AAA ATPase can selectively target a peroxisome for degradation.

It was first assessed whether the loss of the AAA ATPase complex results in a decrease of peroxisome numbers. When HeLa cells were depleted of either PEX1 or PEX26 expression using RNAi, it was found that a significant decrease in peroxisome numbers resulted, whereas the depletion of PEX14, the PEX5 membrane receptor did not cause a significant decrease in peroxisome numbers compared to the non-targeting control siRNA. This loss of peroxisomes upon depleting the cells of AAA ATPase function was not due to the down regulation of biogenesis but rather by their degradation by autophagy. Inhibiting autophagosome formation (using siATG12) prevented the loss of peroxisomes due to AAA ATPase inhibition.

These results indicate that the peroxisomal AAA ATPase is not only involved in the assembly of peroxisomes, but also in the regulation of peroxisome degradation by preventing the signal for peroxisome degradation. Thus, the loss of peroxisomes in the AAA ATPase defective PBD patients may not be due to lack of peroxisome biogenesis, but rather the increase in peroxisome degradation. Thus, since the loss of AAA ATPase function results in the indiscriminating degradation of peroxisomes, inhibiting autophagy by autophagy inhibitor drugs can increase the presence of functional peroxisomes in AAA ATPase defective patient cells.

By increasing peroxisome numbers in PBD patient cells, restoration or improvement of peroxisome function may occur.

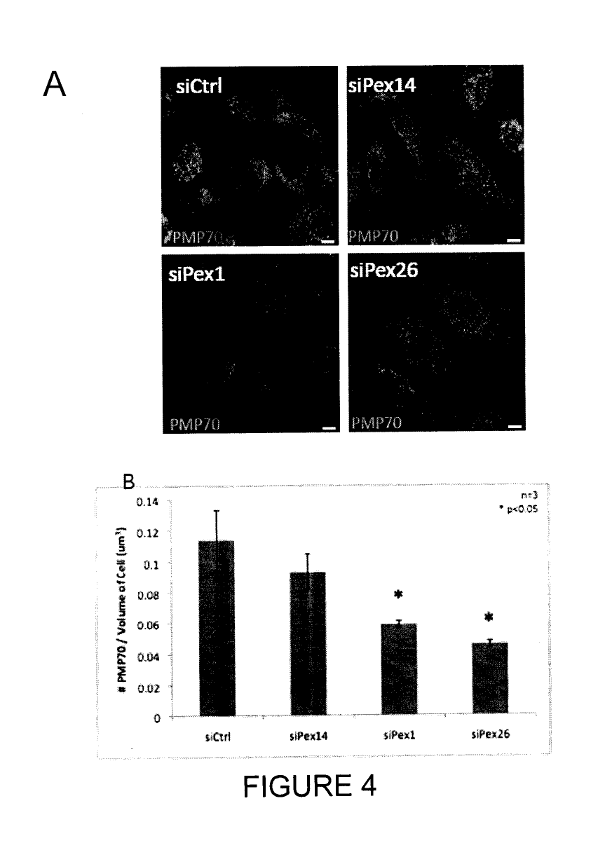

FIG. 4 shows that disruption of the AAA ATPase complex causes a decline in peroxisome numbers. HeLa cells were treated with control siRNA (siCtrl), or against the importer PEX14 (siPEX14), or the AAA ATPase components PEX1 (siPEX1) or PEX26 (siPEX26). Panel (A) shows immunofluorescence image of cells treated with siRNA as indicated. Cells were fixed, permeabilized and stained for a peroxisomal membrane marker PMP70. Note a decrease in PMP70 staining, indicating a decrease in peroxisome numbers. Panel (B) shows a bar graph of the quantification of the number of peroxisomes in the cells normalized against volume of the cells, using Volocity™ (PerkinElmer) particle quantification software. Note that there are fewer peroxisomes in both the siPEX1 and the siPEX26 treated cells compared to control. However, the decrease in the number of peroxisomes in siPEX14 was not significant. The small decrease is likely due to inhibition of biogenesis (data not shown). Data are based on the average of three independent experiments where 100 cells were quantified for each set of experiments. * p<0.05.

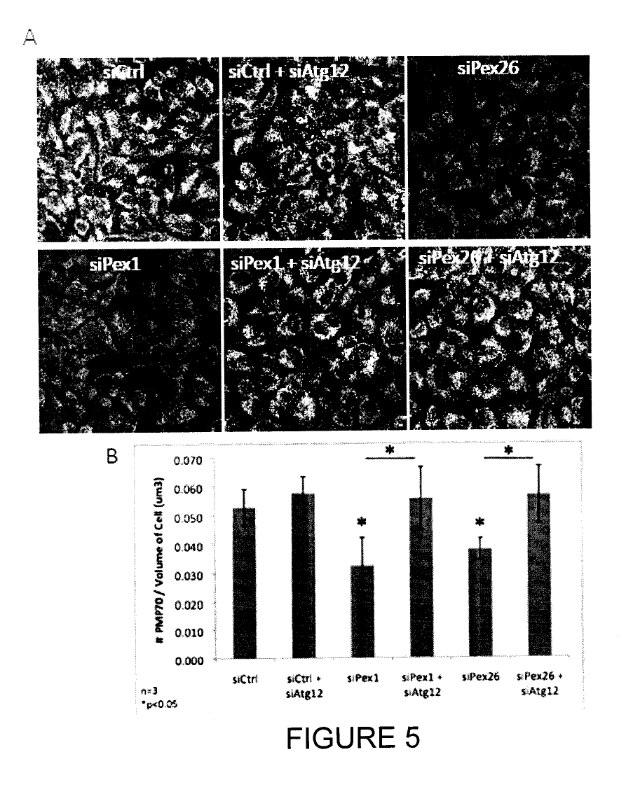

FIG. 5 shows the disruption of autophagy rescues AAA ATPase mediated peroxisome loss. The pathway involved in the formation of autophagosomes was inhibited by the knockdown of the autophagosome factor Atg12. Panel (A) shows immunofluorescent fluorescent images of Hela cells treated with various siRNAi as indicated. Note the decrease in peroxisome numbers in both siPEX26 and siPEX1, but not in those also treated with siAtg12. Panel (B) shows quantification of the number of PMP70 per volume of cell. Note the recovery of the peroxisome numbers upon co-depletion of the autophagy factor Atg12 along with the AAA ATPase component.

### Example 9

### Autophagy Inhibitors

Different autophagy inhibitors were tested: Bafilomycin A1 (referenced interchangeably as “Bafilomycin”), chloroquine, and LY294002.

To demonstrate that inhibiting autophagy increases peroxisome numbers and function in patients with defect their peroxisomal AAA ATPase function, two fibroblast cell lines derived from PEX-p.Ile700fs and PEX1-G843D PBD patients were treated with various known autophagy inhibitors selected from Table 1.

Changes in peroxisomes number upon treatment with autophagy inhibitors was examined using two different techniques. In one method, using immunofluorescent microscopy, cells were probed for the peroxisomal protein PMP70 and the images were analyzed for peroxisome numbers. The change in the amount of the peroxisomal protein PEX14 was also assessed by immunoblot analysis. Evidence for an increase in peroxisome numbers and/or function was observed in both PEX-p.Ile700fs and PEX1-G843D PBD cell lines after 24 hours of drug treatment. The significant increase in peroxisome numbers in the mutant cells and no significant increase in the control cells, indicate that the stabilization of peroxisome degradation may occur upon the inhibition of autophagy. Similarly, the increase in the peroxisomal protein PEX14 after just 24 hours further illustrates that peroxisomes are being stabilized and not degraded.

To determine whether the peroxisome numbers increased with time, peroxisome numbers of cell treated with the autophagy inhibitor LY294002 were examined at 24 and 48 hours. At 48 hours treatment a greater increase in peroxisome number for all cells including control were observed, illustrating that biogenesis of peroxisomes was not affected but instead only the degradation of peroxisomes was inhibited.

Whether autophagy inhibitors can improve peroxisome function was also examined. The ability of autophagy inhibitors to import matrix proteins was gauged to measure functional peroxisomes. The immortalized PEX1-G843D patient cell line (M2H) was used, which stably expresses a fluorescent-tagged matrix marker that is cytosolic at baseline indicating the inability of the cells to import matrix proteins into the peroxisome (Zhang et al., 2010). It was assessed whether autophagy inhibitors could rescue the import defect typically found in PEX1 mutant cells. 24 hours after treatment with autophagy inhibitors, an increase in GFP-SKL positive punctate peroxisomes was found. This demonstrates a corresponding increase in matrix protein import. Previous experiments with the M2H cell line showed that increase in peroxisomal import of the fluorescent reporter coincides with improved peroxisomal biochemical functions (Zhang et al., 2010). The instant experiments of this example illustrate that autophagy inhibitors are able to improve peroxisomal function by stabilizing the presence of mature peroxisomes.

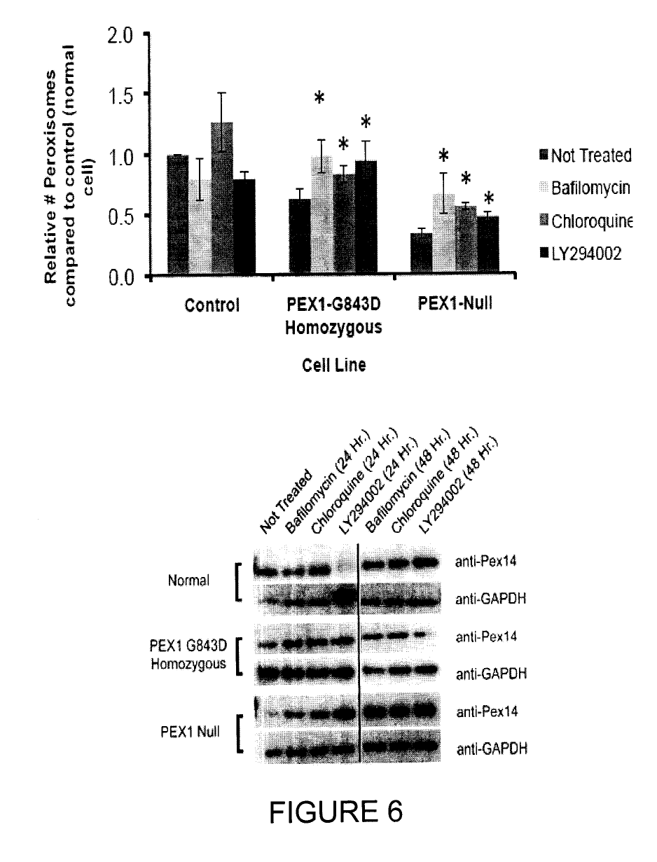

FIG. 6 shows data illustrating that autophagy inhibitors recruit peroxisome numbers. Human fibroblast cells from normal (control), PEX1-G843D homozygous and PEX1 null patient cells were treated with autophagy inhibitor Bafilomycin, Chloroquine or LY294002 for 0, 24 hr. Panel (A) shows a bar graph of number of peroxisomes relative to the non-treated control cells. A significant increase (as determined by student t test) in peroxisome numbers was observer in both PEX1-G843D and PEX1-null mice compared to non-treated cells (* p<0.05). Panel (B) shows immunoblot analysis of the peroxisomal protein PEX14. These data show an increase in PEX14 in patient cell lines when treated with various autophagy inhibitors as indicated. Cells were treated with drugs for either 24 hours or 48 hours. An increase in PEX14 expression is observed for both the PEX1-G843D cells and PEX1-null cells at both 24 and 48 hours. However, in normal cells increase in peroxisomes is only seen after 48 hr. GAPDH served as loading control.

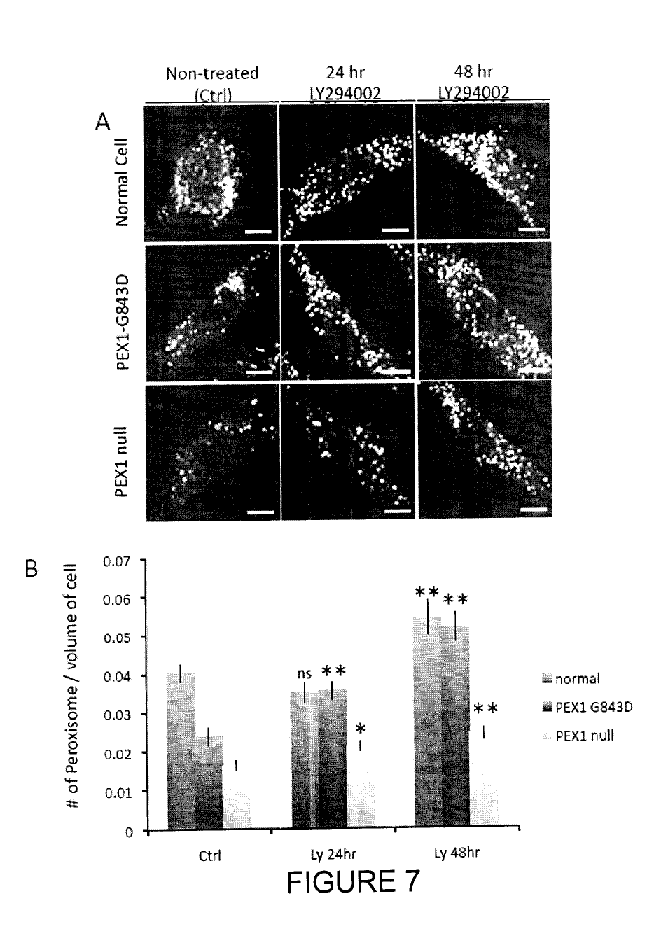

FIG. 7 shows that inhibiting autophagy in PBD patient cells restores peroxisome numbers. Human fibroblast cells from normal, PEX1-G843D homozygous (PBD214) and PEX1 null patient cells were treated with autophagy inhibitor LY294002 for 0, 24 hr and 48 hrs. Panel (A) shows representative images of cells treated with 50 μM LY294002 as indicated. Cells were fixed and immunostained for endogenous PMP70 at 0, 24 and 48 hrs ager treatment. The bar represents 10 μm. Panel (B) shows a graph of the quantification of the number of peroxisomes in the human fibroblast cells. An increase in both the PEX1-G843D cells and PEX1-null cells is observed in both 24 and 48 hours. However, in normal cells, an increase in peroxisomes is only seen after 48 hr. 75 cells were counted over 3 independent experiments. A Student T-Test was performed to compare 24 h and 48 hr to non-treated (0 Hr): * p<0.05, ** p<0.01, ns=not significantly different.

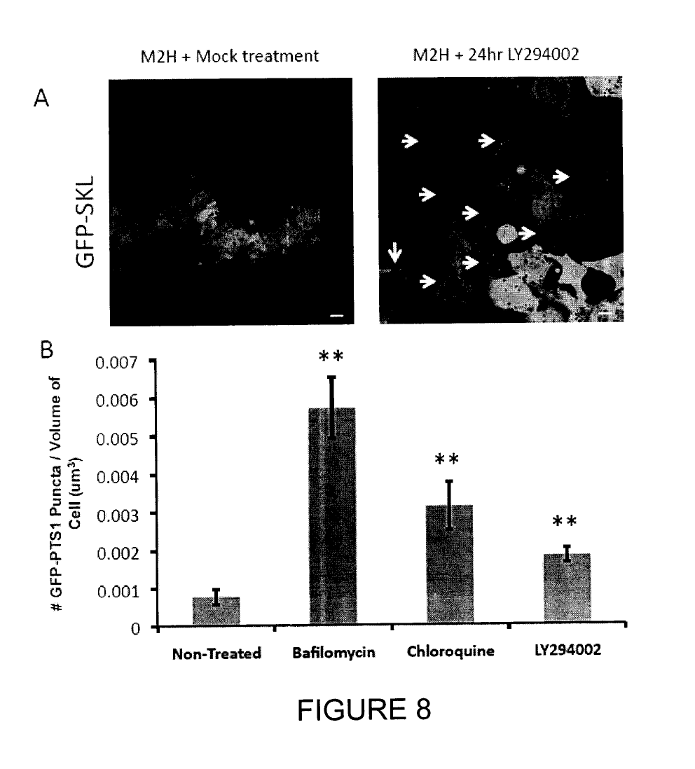

FIG. 8 shows that inhibiting autophagy restores peroxisome function in PBD cells. To determine whether an Autophagy Inhibitor can restore peroxisome function in PBD cells, the import of a matrix marker was examined. There is a correlation between increase in matrix protein import and peroxisomal function. Panel (A) shows M2H, a PEX1-G843D derived cells stably expressing a fluorescent matrix marker, GFP-SKL, was either mock treated or treated with 50 μM LY294002. After 24 hours cells were imaged live. In the LY294002 treated cells an increase in the number of cells with punctate structures were observed (white arrowhead) compared to mock treated cell. Panel (B) shows that the number of peroxisomes in 100 cells were quantified in cells treated with Bafilomycin, Chloroquine or LYS94002. For all treatments significant increase in peroxisome number was observed in cells treated with any of the inhibitors (**p<0.001). Scale bar=10 μm.

### Example 10

### 1-((2-(diethylamino)ethyl)amino)-4-methylthioxanthen-9-one

The structure of 1-((2-(diethylamino)ethyl)amino)-4-methylthioxanthen-9-one, also referred to herein as lucanthone (or Miracil D) is shown in Formula VIII, a known autophagy inhibitor.

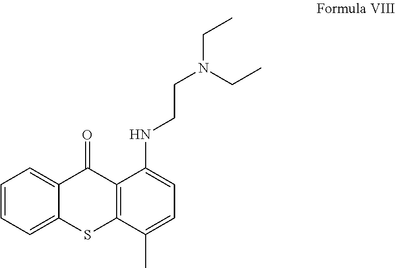 [SMILES: CCN(CC)CCNc1ccc(C)c2sc3ccccc3c(=O)c12]

### Example 11

### 1-(2-diethylaminoethylamino)-4-(hydroxymethyl)-9-thioxanthenone

The structure of 1-(2-diethylaminoethylamino)-4-(hydroxymethyl)-9-thioxanthenone, also known as hycanthone, is shown in Formula IX, a known autophagy inhibitor.

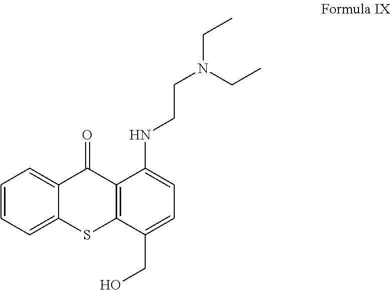 [SMILES: CCN(CC)CCNc1ccc(CO)c2sc3ccccc3c(=O)c12]

### Example 12

### N-[[1-[[2-(diethylamino)ethyl]amino]-9-oxo-9H-thiaxanthen-4-yl] methyl]methanesulfonamide

The structure of N-[[1-[[2-(diethylamino)ethyl]amino]-9-oxo-9H-thiaxanthen-4-yl]methyl]methanesulfonamideis, which may be referenced herein as WIN-33377 or SR-233377, is shown in Formula X, a known autophagy inhibitor.

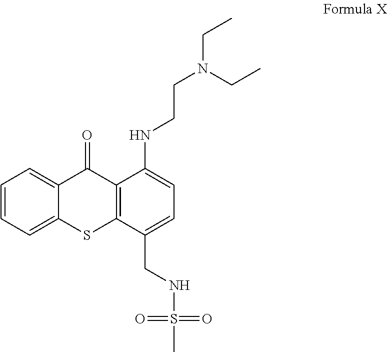 [SMILES: CCN(CC)CCNc1ccc(CNS(C)(=O)=O)c2sc3ccccc3c(=O)c12]

### Example 13

### AAA ATPase-Deficient Peroxisome Degradation for Treatment of Peroxisome Biogenesis Disorders e

Overview:

The quality control mechanisms used for controlling peroxisome number in the cell include an intricate balance between peroxisomal biogenesis and degradation (pexophagy). 90% of Peroxisome Biogenesis Disorders (PBDs) are due to mutations encoding for the peroxisomal AAA ATPase complex (PEX1, PEX6 and PEX26), which is responsible for removing ubiquitinated proteins from the peroxisomal membrane. Acutely depleting cells of PEX1 and PEX26 results in ROS and ubiquitination that results in the induction of autophagy and pexophagy, respectively. Inhibiting autophagy in PEX1-mutated PBD patient cell lines results in a recovery of peroxisome number, matrix protein import, and β-oxidation.

Materials and Methods

Plasmids.

GFP-LC3 was a gift from Dr. Yoshinori Ohsumi (National Institute of Basic Biology, Okazaki, Japan). GFP-Ub(G76V) (GFP-Ub) was purchased from Addgene. pmRFP-SKL (RFP-SKL) was constructed by using the oligonucleotides Fp 5′-CGGGATCCACCGGTCGCCACCATG-3′ and Rp 5′-CAGCGGCCGCTTAAAGCTTGG AGGCGCCGGTGGAGTGGCG-3′ and pmRFP-N1 as template DNA. The resulting products were digested with BamHI and NotI, and then ligated into pmRFP-N1. The plasmid was sequenced for confirmation (The Centre for Applied Genomics, Ontario, Canada). Primers were produced by Sigma Genosys (Ontario, Canada) and Invitrogen. The cloning strategies and primer sequences used are available upon request.

Reagents.

Leupeptin (Bioshop, or Enzo Life Sciences) was used at 0.5 mM and E-64 (Enzo Life Sciences) at 2 μM. OxyBURST® Green H2DCFDA succinimidyl ester (2′,7′-dichlorodihydrofluorescein diacetate, SE) (Oxyburst) was obtained from Life Technologies and used at 10 μM. Chloroquine, Bafilomycin, Difluoromethylornithine (DFMO), Clomipramine and MG-132 were obtained from Sigma. LY294002 was obtained from Cedarlane. N-ethylmaleimide (NEM) was kindly obtained from the Dr. Daniela Rotin lab (Hospital for Sick Children, Toronto, Ontario). N-acetylcysteine (NAC) was obtained from The University of Toronto MedStore (Toronto, Ontario) and was used at 20 μM.

siRNA.

siRNA targeting non-targeting controls (proprietary sequence) was obtained from Sigma (Texas, USA). siRNA targeting PEX26 (CAAGACCCAGCCAA UCAAATT), ATG12 (GUGGGCAGUAGAGCGAACATT), PEX1 (CCAAGCAACUU CAGUCAAATT), PEX14 (GAACUCAAGUCCGAAAUUTT), and NBR1 (GGAGUGGA UUUACCAGUUAUU) were produced by GenePharma (Shanghai, China). shRNA targeting PEX1 and PEX14 have the same sequences as previously mentioned. Individual pGIPZ clones were found through GE Healthcare Dharmacon Inc. (Ontario, Canada) and were obtained through The Hospital for Sick Children SPARC BioCentre (Ontario, Canada).

Antibodies.

Primary antibodies used in this study include: rabbit polyclonal anti-catalase (EMD Biosciences, Calbiochem, Darmstadt, Germany); rabbit monoclonal anti-PMP70 (Epitomics, an Abcam Company, CA, USA); rabbit test bleed anti-PEX5 (Cedarlane); mouse monoclonal anti-HA (Covance, New Jersey, USA); rabbit polyclonal anti-HA (Santa Cruz Biotechnology, Inc., Texas, USA); rabbit polyclonal anti-PEX14 (Merck Millipore, Darmstadt, Germany); and mouse monoclonal anti-GAPDH-HRP (Novus Biologicals, Ontario, Canada). The anti-LC3 antibody was a gift from Dr. John Brumell. Secondary antibodies used for Western Blotting include goat anti-rabbit IgG-HRP (Santa Cruz Biotechnology, Inc.) and goat anti-mouse IgG-HRP (Cedarlane). Secondary antibodies used for immunofluorescence include goat anti-rabbit IgG, DyLight™ 594 and goat anti-mouse IgG, Dylight™ 488 (Thermo Scientific, Illinois, USA).

Cell Culture. HeLa cells were obtained from American Type Culture Collection (ATCC). Wild-type and ATG5−/− MEFs were obtained from the Dr. John Brumell lab (Hospital for Sick Children, Toronto, Ontario). All PBD patient cell lines (PEX1-Null, PEX1-G843D Homozygous and Hemizygous, PEX1-G843D-PTS1) were obtained from the Dr. Nancy Braverman lab (McGill University, Montreal, Quebec). The HEK293 cell line expressing HA-Ub was obtained from the Dr. Brian Rought lab (MaRS, Toronto, Ontario).

HeLa, MEFs, HEK293s, control fibroblasts, and the PEX1-G843D-PTS1 fibroblasts were grown in DMEM (Thermo Scientific) supplemented with 10% fetal bovine serum (FBS; Gibco, Life Technologies, Ontario, Canada) and 0.1% 200 mM L-glutamine (Thermo Scientific). PEX1-Null and PEX1-G843D Homozygous fibroblasts were grown in DMEM supplemented with 15% FBS and 0.1% 200 mM L-glutamine. All cells were cultured at 5% CO₂ in a 37° C. incubator.

For immunofluorescence, cells were grown either in LabTek chamber slides or on #1 glass coverslips. Plasmids and siRNA were transfected using Optimem (Gibco) and Lipofectamine-2000 (Invitrogen) according to the manufacturer's instructions. For simultaneous siRNA knockdown and plasmid overexpression, siRNA alone was transfected on the first day, and siRNA and plasmids were transfected simultaneously on the second day, using Lipofectamine-2000. Cells were collected or imaged two days after plasmid/siRNA transfection.

For live cell imaging of Oxyburst, cells were grown, manipulated, imaged in LabTek chamber slides (Nalgene Nunc, New York, USA). Prior to live cell imaging, the media was changed to CO2-independent medium (Gibco), and Oxyburst was added at a final concentration of 10 μM.

Microscopy.

Laser-scanning confocal microscopy was performed on a Zeiss LSM710 with a 63×/1.4 Plan-Apochromat oil objective. When required, images were acquired in Z-sections of 0.5 to 1.0 μM thickness. Live cell imaging was performed at 37° C.

For immunofluorescence, cells were fixed using 3.7% paraformaldehyde (Electron Microscopy Sciences) in PBS for 15 minutes and permeabilized using 0.1% Triton X-100 (Fisher Scientific) in PBS for 20 minutes. Cells were then incubated in blocking buffer and incubated with the appropriate primary and secondary antibodies for 2 hours (or overnight at 4° C.), and 1 hour, respectively.

Images were analyzed using Volocity software (Perkin Elmer). At least 30 cells were quantified and averaged per experiment, and were normalized against the control. For quantification of peroxisome density, the number of PMP70-stained or GFP-LC3 or GFP-PTS1 structures was divided by the volume of each cell, and the averages were normalized to the respective controls. For quantification of the total PMP70 intensity, the total fluorescence intensity of the red channel was divided by the volume of each cell. For quantification of the individual PMP70 puncta fluorescence intensity, the fluorescence of each PMP70-positive structure was divided by the volume of each cell. Oxyburst fluorescence intensity was determined by the total fluorescence of each cell.

Western Blots.

Cells were lysed with 100 mM Tris pH 9 containing 1% SDS (Bio-Rad) and protease inhibitor cocktail (Sigma), and the lysate was heated at 95° C. with vortexing for 30 minutes, then centrifuged at 13,000×g for 10 minutes. The protein concentration in the supernatant was determined by BCA assay (VWR), equivalent sample amounts were subjected to SDS-PAGE, and protein was transferred to 0.45 μm PVDF membrane (BioRad) and probed with the appropriate primary and HRP-conjugated secondary antibodies following standard protocols. Blots were developed using either Amersham ECL Prime Western Blot Detection Reagent (GE Healthcare Life Sciences) or EZ-ECL (Biological Industries). Densitometry was performed using ImageJ software.

MTT Assay.

Cells were seeded on a 96-well plate and treated with the specified drugs for the specified period of time. Media would be changed on a daily basis, and replenished with fresh media supplemented with the respective drug. Following the drug treatment time period, cells were treated with the tetrazolium dye MTT 3-(4,5-dimethylthiazol-2-yl)-2,5-diphenyltetrazolium bromide at a final concentration of 0.5 mg/mL, and incubated for 4 hours at 5% CO₂ in a 37° C. incubator. The formazan was dissolved in DMSO. The quantity of formazan was measured by recording the absorbance values at 540 nm using a plate reading spcetrophotometer.

Immunoprecipitation.

HEK293 cells expressing HA-Ub were seeded on 6-well plates. siRNA knockdowns were preformed twice in a 24-h interval. HA-Ub expression was induced by incubating cells with Tamoxifen at 20 μM. Prior to collection, cells were incubated with MG-132 at 10 μM for 4 hours. Cells were collected using 200 μL “IP buffer” (50 mM Tris pH 7.4, 150 mM NaCl, 1 mM EDTA, 1% NP-40) containing 1× protease inhibitor cocktail and NEM at 10 mM. Lysates were spun at 15,000 rpm for 15 minutes at 4° C. 10% of the supernatant was kept as “input.” Mouse monoclonal anti-HA was added at a concentration of 1:150 and gently agitated overnight at 4° C. Washed Protein G Sepharose™ 4 Fast Flow (GE Healthcare) beads were added to each reaction and incubated for 2-3 hours with gentle agitation at 4° C. Cells were washed with IP buffer 3-4 times. 2×LSB was added, beads were boiled for 5 minutes at 95° C., and vortexed.

Results and Conclusion

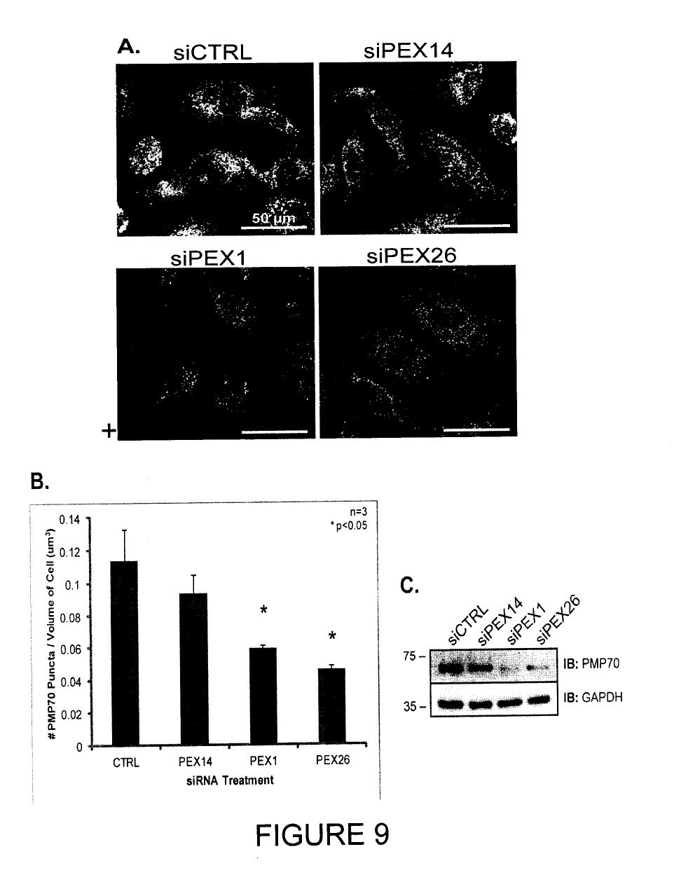

FIG. 9 shows that depletion of the AAA ATPase complex results in a reduction of peroxisome number. In FIG. 9 (Panel A) HeLa cells were transfected with non-targeting siRNA (siCTRL), and siRNA against PEX14, PEX1, and PEX26 as indicated over two consecutive days. Two days later, cells were fixed and immunostained for PMP70. Scale bars, 50 μM. FIG. 9 (Panel B) shows a graph of the number of PMP70 puncta per volume of each cell (um³) of at least 30 cells per trial in (Panel A). The average (n=3) for each condition is shown. Asterisks represent p-values: * p<0.05. In FIG. 9 (Panel C) total cell lysates were prepared from siRNA treated cells (as in Panel A) and immunoblotted with PMP70 and GAPDH antibodies.

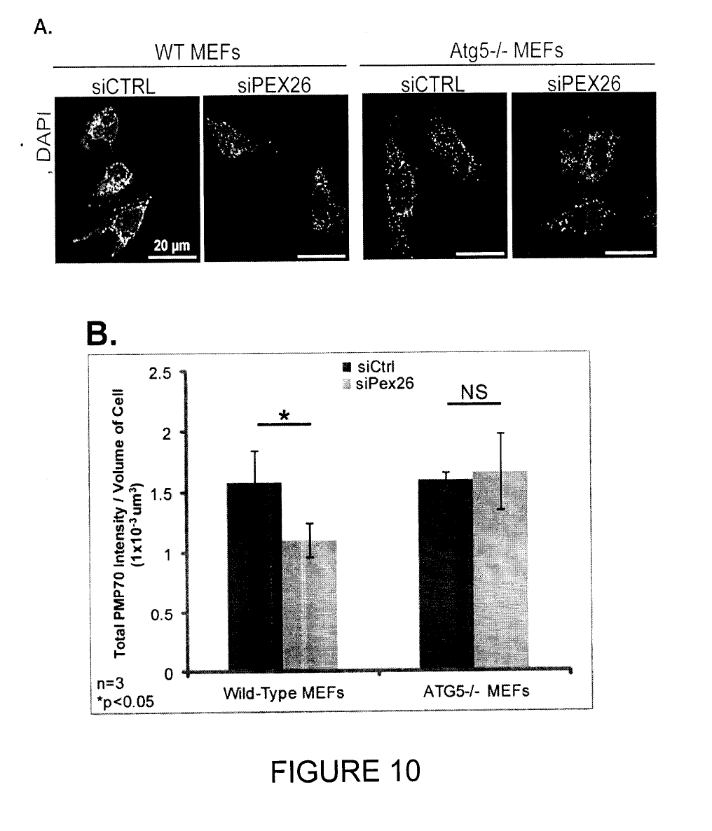

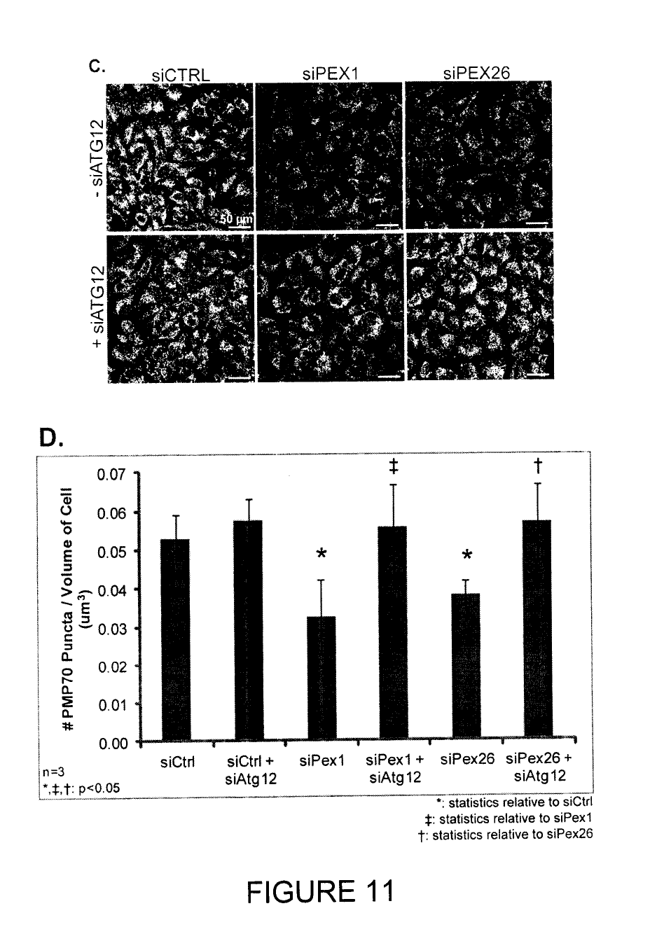

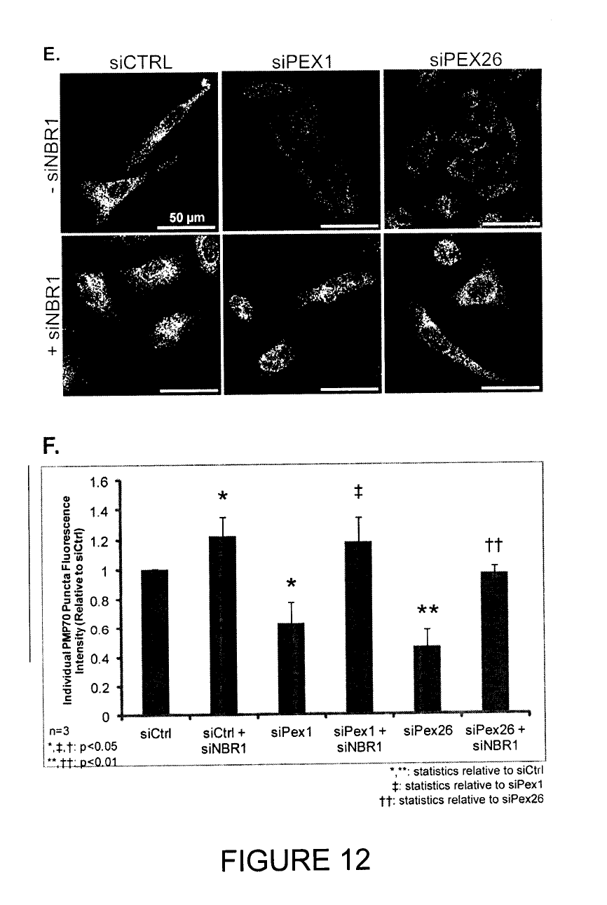

FIG. 10, FIG. 11 and FIG. 12 show that the loss of peroxisomes seen in AAA ATPase-deficient cells is due to pexophagy. In FIG. 10 (Panel A) Wild-type (WT) and ATG5−/− MEFs were transfected with non-targeting siRNA (siCTRL), and siRNA against PEX26 as indicated over two consecutive days. Two days later, cells were fixed and immunostained for PMP70 and DAPI. Scale bars, 20 μM. FIG. 10 (Panel B) is a graph of the total PMP70 intensity per volume of each cell (um³) of at least 30 cells per trial in (Panel A). The average (n=3) for each condition is shown. Asterisks represent p-values: * p<0.05. NS represents non-significance. In FIG. 11 (Panel C), HeLa cells were transfected with non-targeting siRNA (siCTRL), siRNA against PEX1, siRNA against PEX26, all +/−siRNA against ATG12 as indicated over two consecutive days. Two days later, cells were fixed and immunostained for PMP70. Scale bars, 50 μM. FIG. 11 (Panel D) is a graph of the number of PMP70 puncta per volume of each cell (um³) of at least 30 cells per trial in (Panel C). The average (n=3) for each condition is shown. Asterisks represent p-values of statistics relative to siCTRL: * p<0.05. Double crosses represent p-values of statistics relative to siPEX1: ‡ p<0.05. Single crosses represent p-values of statistics relative to siPEX26: † p<0.05. In FIG. 12 (Panel E) HeLa cells were transfected with non-targeting siRNA (siCTRL), siRNA against PEX1, siRNA against PEX26, all +/−siRNA against NBR1 as indicated over two consecutive days. Two days later, cells were fixed and immunostained for PMP70. Scale bars, 50 μM. FIG. 12 (Panel F) is a graph of the individual PMP70 puncta fluorescence per volume of each cell (um³) normalized to siCTRL of at least 30 cells per trial in FIG. 12 (Panel E). The average (n=3) for each condition is shown. Asterisks represent p-values of statistics relative to siCTRL: * p<0.05, ** p<0.01. Double crosses represent p-values of statistics relative to siPEX1: p<0.05. Single crosses represent p-values of statistics relative to siPEX26: tt p<0.01.

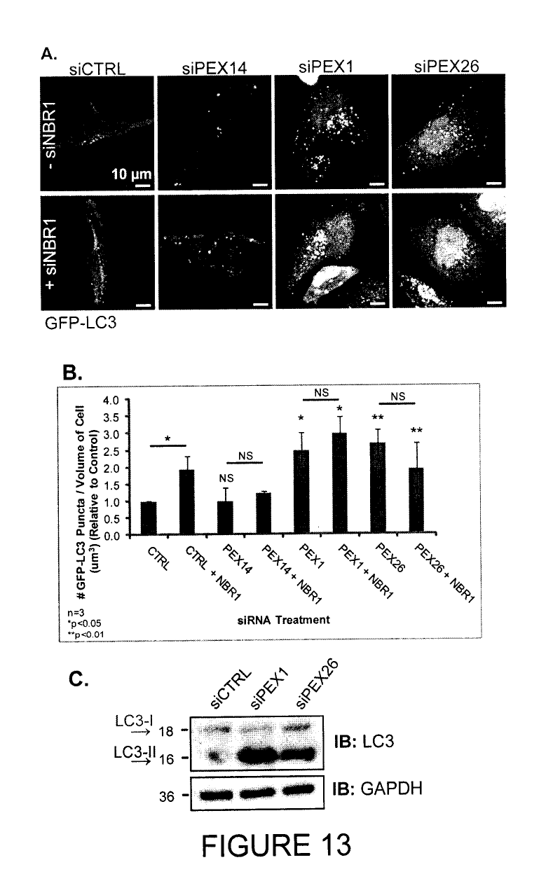

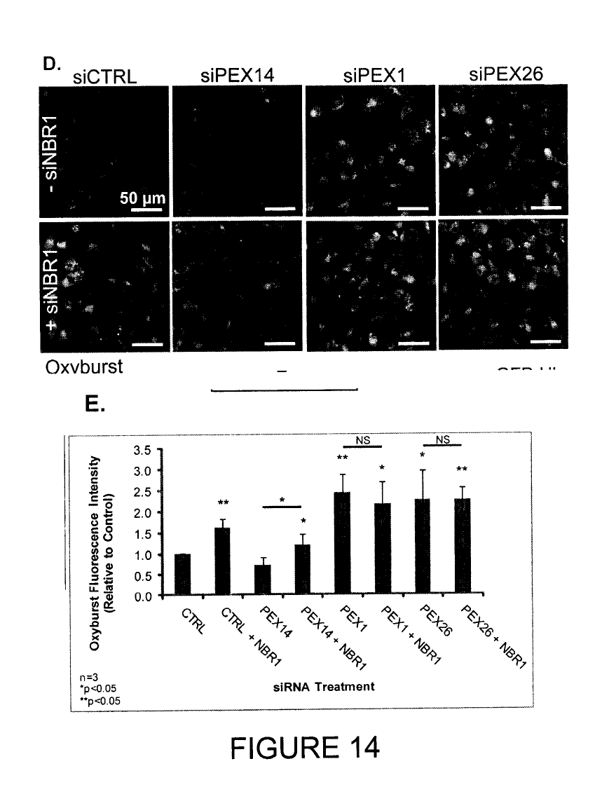

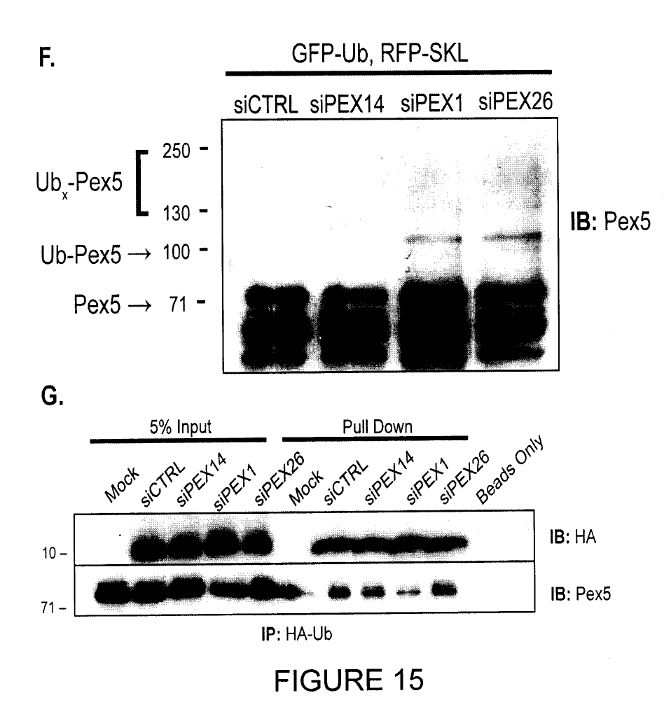

FIG. 13, FIG. 14 and FIG. 15 illustrate a mechanism of AAA ATPase-deficient peroxisome degradation. In FIG. 13 (Panel A) HeLa cells were transfected with GFP-LC3 and non-targeting siRNA (siCTRL), siRNA against PEX1, siRNA against PEX14, siRNA against PEX26, all +1-siRNA against NBR1 as indicated over two consecutive days. Two days later, cells were fixed. Scale bars, 10 μM. FIG. 13 (Panel B) shows a graph of the number of GFP-LC3 puncta per volume of each cell (um³) normalized to siCTRL of at least 30 cells per trial in Panel A. The average (n=3) for each condition is shown. Asterisks represent p-values: * p<0.05, **p<0.01. NS represents non-significance. In FIG. 13 (Panel C) total cell lysates were prepared from HeLa cells treated with non-targeting siRNA, or siRNA against PEX1 or PEX26 as well as with E-64 and leupeptin inhibitors. Lysates were immunoblotted with LC3 and GAPDH to confirm LC3-II increase seen. In FIG. 14 (Panel D) HeLa cells were transfected with non-targeting siRNA (siCTRL), siRNA against PEX1, siRNA against PEX14, siRNA against PEX26, all +1-siRNA against NBR1 as indicated over two consecutive days. Two days later, cells were treated with Oxyburst (C₂=10 μM) and were imaged live. Scale bars, 50 μM. FIG. 14 (Panel E) shows a graph of the Oxyburst fluorescence intensity normalized to control of at least 30 cells per trial in Panel D. The average (n=3) for each condition is shown. Asterisks represent p-values: * p<0.05, **p<0.01. NS represents non-significance. In FIG. 15 (Panel F) total cell lysates were prepared from cells transfected with GFP-Ub and RFP-SKL in a 1:4 ratio, and with non-targeting siRNA (siCTRL), or siRNA against PEX14, PEX1, or PEX26, and immunoblotted with Pex5. Asterisks represent p-values: * p<0.05, **p<0.01. In FIG. 15 (Panel G) immunoprecipitation of HA (HA-Ub) is conducted from HEK293 cells expressing HA-Ub under the control of a tamoxifen-inducible promoter that have been treated with siRNA against non-targeting siRNA, or siRNA against PEX14, PEX1, or PEX26. Mock indicates cells whereby no tamoxifen was added (no HA-Ub induction). 5% input, pull downs, and antibody-only and beads-only controls were immunoblotted for HA and Pex5.

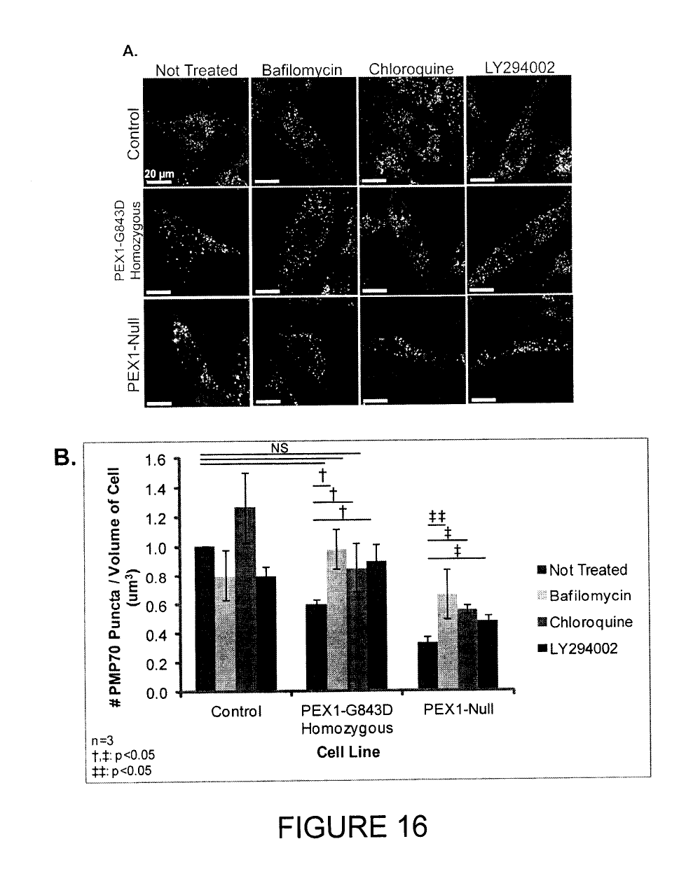

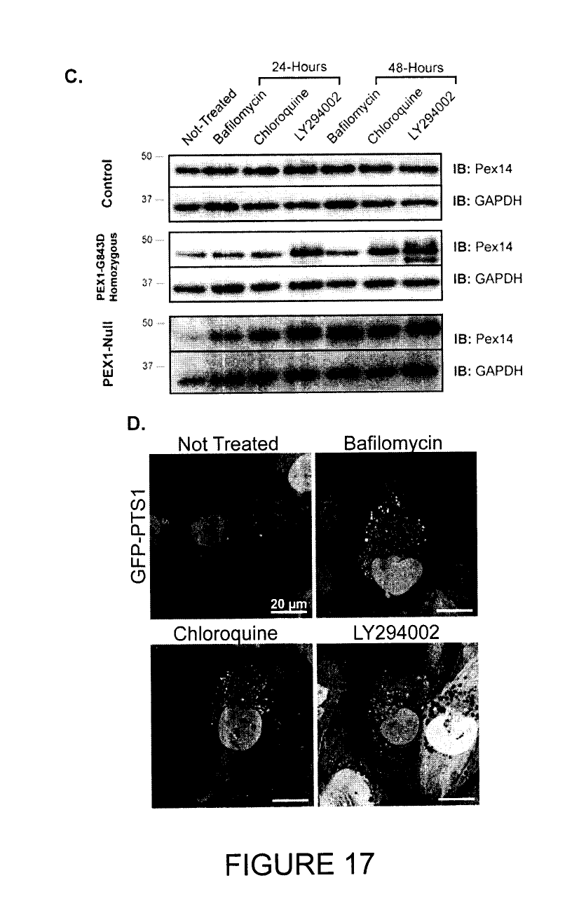

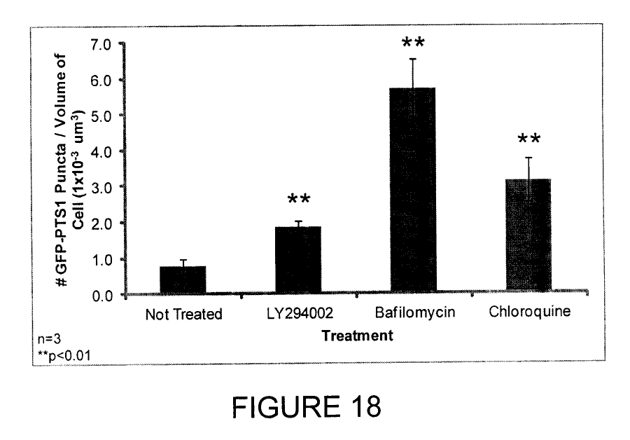

FIG. 16, FIG. 17 and FIG. 18 show that Autophagy inhibitors recover peroxisome number and function in PEX1-mutated PBD fibroblasts. In FIG. 16, (Panel A) control, PEX1-G843D homozygous, and PEX1-null fibroblasts were treated with Bafilomycin (C₂=2 nM), Chloroquine (C₂=20 μM), LY294002 (C₂=5 mM). After 24-hours, cells were fixed and immunostained for PMP70. Scale bars, 20 μM. FIG. 16, (Panel B) shows a graph of the number of PMP70 puncta per volume of each cell (um³) normalized to the non-treated control fibroblasts of at least 30 cells per trial in (A). The average (n=3) for each condition is shown. Single crosses represent p-values of statistics relative to non-treated PEX1-G843D homozygous fibroblasts: † p<0.05. Double crosses represent p-values of statistics relative to non-treated PEX1-null fibroblasts: ‡ p<0.05, ‡‡ p<0.01. NS indicates non-significance relative to non-treated control fibroblasts. In FIG. 17 (Panel C) total cell lysates prepared from fibroblasts as in FIG. 16 (Panel A) treated with the various drugs for 24- and 48-hours and immunoblotted with Pex14 and GAPDH. In FIG. 17 (Panel D) PEX1-G843D homozygous fibroblasts stably expressing GFP-PTS1 (PEX1-G843D-PTS1) were treated with various drugs as in FIG. 16 (Panel A). Scale bars, 20 μM. FIG. 18 shows a graph of the number of GFP-PTS1 puncta per volume of cell (1×10⁻³ μM) of at least 30 cells per trial in Panel D. The average (n=3) for each condition is shown. Asterisks represent p-values relative to non-treated cells: ** p<0.01.

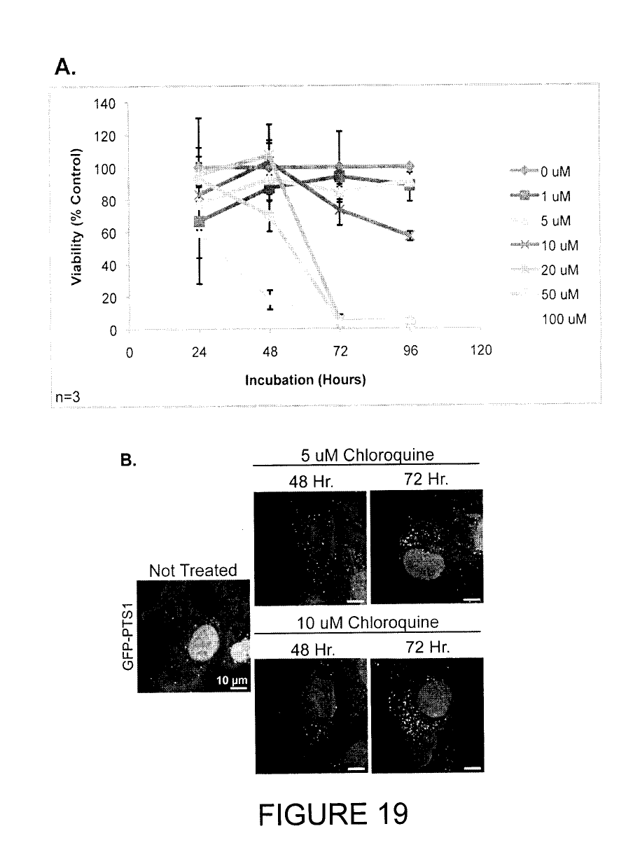

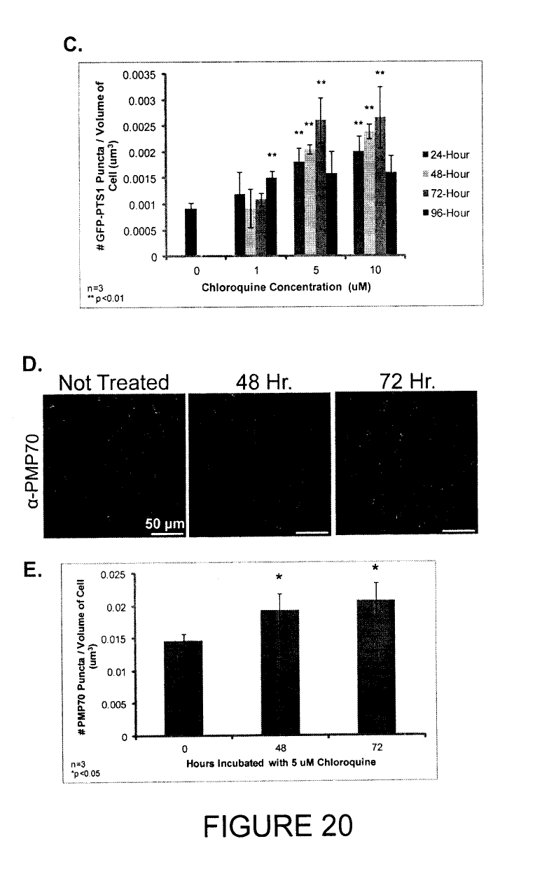

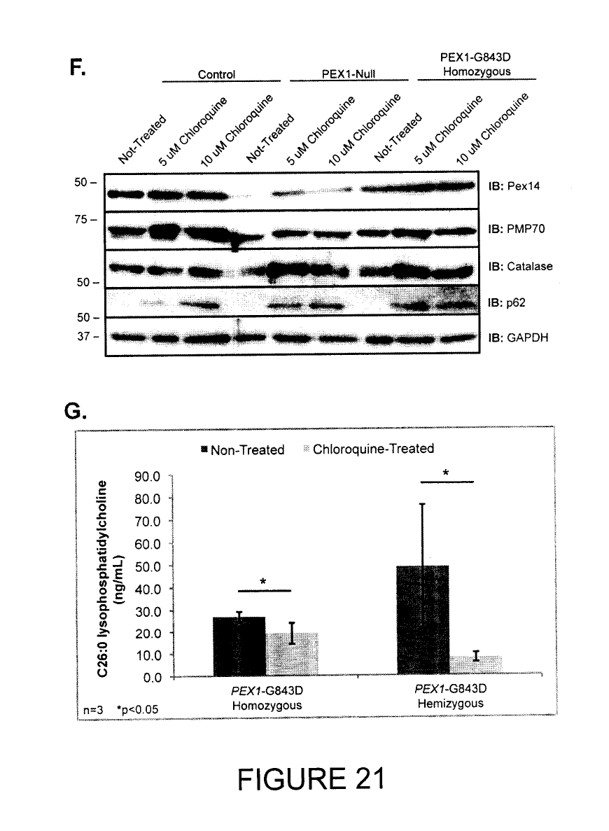

FIG. 19, FIG. 20 and FIG. 21 indicate that the FDA-approved drug chloroquine recovers peroxisome number and function without compromising cellular viability. In FIG. 19 (Panel A) control fibroblasts that were treated with varying concentrations of chloroquine (0, 1, 5, 10, 20, 50, 100 μM) for 24-, 48-, 72-, and 96-hours. Fresh drug was added each day. Viability was determined by MTT assay, and absorbance values were normalized to not-treated cells to obtain the viability (% control). In FIG. 19, (Panel B) PEX1-G843D-PTS1 cells were not-treated, or treated with 5 or 10 μM Chloroquine for 48- or 72-hours, and fixed. Scale bars, 10 μM. FIG. 20 (Panel C) shows a graph of the number of GFP-PTS1 puncta per volume of each cell (um³) normalized to the non-treated fibroblasts of at least 30 cells per trial (in Panel B of FIG. 19). The average (n=3) for each condition is shown. Asterisks represent p-values of statistics relative to non-treated fibroblasts: ** p<0.01. In FIG. 20, (Panel D) PEX1-G843D-PTS1 fibroblasts were not-treated, or treated with 5 μM Chloroquine for 48- or 72-hours as indicated. Cells were fixed and immunostained for PMP70. FIG. 20 (Panel E) is a graph of the number of PMP70 puncta per volume of each cell (um³) of at least 30 cells per trial (in Panel D). The average (n=3) for each condition is shown. Asterisks represent p-values of statistics relative to non-treated fibroblasts: * p<0.05. In FIG. 21, (Panel F) total cell lysates were prepared from control, PEX1-null, and PEX1-G843D fibroblasts treated with 5 or 10 μM Chloroquine as indicated for 72-hours, and immunoblotted with Pex14 and GAPDH antibodies. FIG. 21, (Panel G) is a graph of functional recovery in PEX1-G843D homozygous and hemizygous primary fibroblasts cultured in regular media or regular media+chloroquine for 144-hours (6 days). VLCFA was measured as C26:0 lysophosphatidylcholine. The average (n=3) for each condition is shown. Astericks represent p-values of statistics relative to non-treated fibroblasts: * p<0.05.

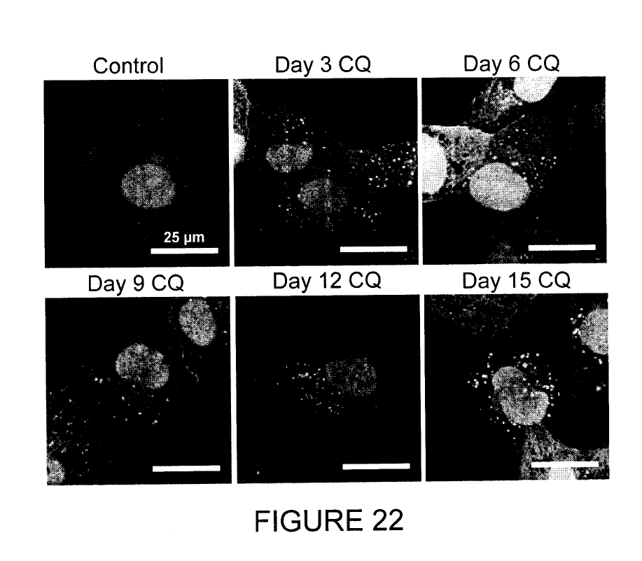

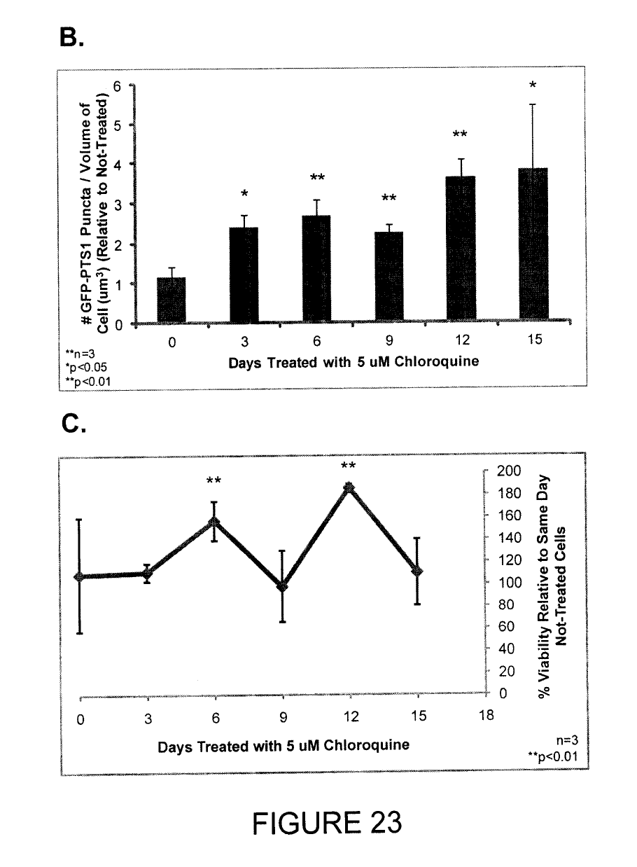

FIG. 22 and FIG. 23 illustrate that chloroquine may be used for PBD patients to improve peroxisomal function. PEX1-G843D-PTS1 fibroblasts were treated with 5 μM Chloroquine for 15 days. Media was replenished with fresh drug each day. Every 3 days, cells were seeded and either fixed for imaging, or treated with MTT to determine viability. FIG. 22 provides representative images of PEX1-G843D-PTS1 fibroblasts treated with 5 μM Chloroquine on days 0 (control), 3, 6, 9, 12, and 15. Scale bar, 25 μM. FIG. 23 (Panel B) is a graph of the number of GFP-PTS1 puncta per volume of each cell (um³) normalized to the non-treated fibroblasts of at least 30 cells per trial in (A). The average (n=3) for each condition is shown. Asterisks represent p-values of statistics relative to non-treated fibroblasts: * p<0.05, ** p<0.01. FIG. 23 (Panel C) is a graph of the % viability relative to not-treated cells. The average (n=3) for each condition is shown. Asterisks represent p-values of statistics for the Chloroquine-treated relative to the not-treated fibroblasts for each day: ** p<0.01.

These data illustrate that depletion of the AAA ATPase complex results in a reduction of peroxisome number. AAA ATPase-deficient peroxisomes exhibit a decline in peroxisome number due to peroxisomes being selectively targeted for degradation via the autophagy pathway. Together, these results indicate that in AAA ATPase-deficient peroxisomes, autophagy may be upregulated in a ROS-dependent manner. Furthermore, since a co-depletion of NBR1 in siPEX1 and siPEX26 cells results in a maintenance of ROS and LC3 levels, this indicates that autophagy and pexophagy may be independently induced and upregulated.

In this Example, the mechanism of AAA ATPase-deficient peroxisome degradation in a mammalian cell system is revealed. More specifically, it has been shown that not all PEX genes are solely involved in the biogenesis of peroxisomes as once believed. Rather, some PEX genes (namely, PEX1 and PEX26, and possibly PEX6) are involved in peroxisomal quality control by preventing the degradation of functional peroxisomes. The AAA ATPase complex may not only be involved in the assembly of peroxisomes, but also in the regulation of pexophagy, by preventing the signal for peroxisome degradation. The finding that the AAA ATPase complex may have a dual role, both in the biogenesis of peroxisomes, but also in the regulation of peroxisome degradation, has not previously been suggested or reported.

Indeed, in cells depleted of PEX1 or PEX26, there is a reduced peroxisome number. This results in reactive oxygen species (ROS), which increases autophagy induction. This also results in an accumulation of ubiquitinated-Pex5, and as a result, pexophagy induction. Both of these pathways eventually converge, resulting in peroxisome degradation. These autophagy and pexophagy pathways are distinct in terms of their induction since when cells are depleted of PEX1 or PEX26 an increase in GFP-LC3 and ROS can be seen, that is maintained when cells are also co-depleted of NBR1. Since pexophagy feeds into the autophagy pathway, these distinct pathways both converge to result in peroxisomal degradation. These results indicate that PBDs caused by AAA ATPase complex mutations may in fact be due to an increase in peroxisomal degradation, as opposed to a defect in peroxisome biogenesis. As such, the recovery of peroxisome number and function in PEX1-mutated fibroblasts in a therapeutic manner by preventing peroxisome degradation, was evaluated using autophagy inhibitors.

Autophagy Inhibitors Recover Peroxisome Number and Function in PEX1-Mutated PBD Fibroblasts.

Since the intermediate-mild phenotypes of PBDs cause a progressive loss of clinical function over time, it was evaluated whether these patients could benefit from therapeutic interventions that restore peroxisomal number and function. Chemical chaperones, such as trimethylamine N-oxide (TMAO) and betaine can be used to stabilize the PEX1-G843D mutation protein allowing for the assembly of functioning peroxisomes (Zhang et al., 2010). However, PEX1-G843D makes up only 30% of all reported cases of PBDs. Thus, while these drugs may help stabilize the highly unstable PEX1-G843D mutant, it is unlikely that it can be used in other AAA ATPase mutations resulting from deletion of the gene. Furthermore, chemical chaperones non-selectively stabilize mutant proteins, which could lead to negative effects throughout the cell long-term. It was evaluated whether downregulating autophagy at a sub-clinical level in PEX1-null and PEX1-G843D cell lines obtained from PBD patients could restore peroxisome number and function.

In order to investigate this, 4 PBD patient fibroblast cell lines were obtained, which have been previously reported (Zhang et al., 2010): 1) a PEX1-null cell line, 2) PEX1-G843D homozygous and hemizygous cell lines, which have a missense allele that accounts for ⅓ of all ZSD alleles, and 3) a PEX1-G843D-PTS1 cell line, where GFP-PTS1 is stably expressed. These cell lines were treated, along with a control fibroblast cell line, with various autophagy inhibitors that target different points of the autophagy pathway. This includes: LY294002, a PI3K inhibitor that targets autophagosome formation; Bafilomycin, a vacuolar-type H(+)-ATPase inhibitor that targets autophagolysosome formation; and Chloroquine, a lysosomal lumen alkalizer that targets the end of the pathway (reviewed in (Yang et al., 2013)). By treating these cell lines with these inhibitors, the number of peroxisomes was quantified following immunostaining for PMP70, and the results verified using immunoblot analysis. As well, peroxisomal matrix PTS1 import was examined using the PEX1-G843D-PTS1 line.

Treatment of the PEX1-mutated fibroblasts with autophagy inhibitors for 24-hours resulted in a significant increase in peroxisome number (as seen by PMP70 puncta) in both the PEX1-G843D homozygous and PEX1-null cell lines (FIG. 16, panels A and B). Of note, no significant difference in the number of PMP70 puncta was evident between the control fibroblast cell line and the PEX1-G843D cell line when treated with all autophagy inhibitors. This was verified by immunoblot analysis for Pex14 and GAPDH (loading control), where an increase in Pex14 was seen following 24- and 48-hour treatments with autophagy inhibitors (FIG. 17, Panel C).

Subsequently, it was asked whether import function could be improved, in addition to peroxisome number, in these PEX1-mutated fibroblasts. It has been previously shown that in the PEX1-G843D-PTS1 cell line, the GFP-PTS1 is primarily cytosolic, suggesting disrupted matrix protein import (Zhang et al., 2010), and perhaps a leakage of matrix proteins through the transient pore. Furthermore, it has been demonstrated that matrix proteins can still be imported into peroxisomes lacking the AAA ATPase complex (albeit at lower levels) (Zhang et al., 2010), suggesting matrix proteins are able to target efficiently, and it is indeed leakage.

When these PEX1-G843D-PTS1 fibroblasts are treated with various autophagy inhibitors for 24-hours, a significant redistribution of GFP-PTS1 from the cytosol to punctate structures was seen, suggesting a recovery of peroxisomal PTS1 matrix protein import (FIG. 17, Panel D; FIG. 18). This means that not only does inhibiting autophagy result in increased peroxisome membrane structures, but that these peroxisomes may also function in a sense of PTS1 matrix protein import.

The question remains, however, how would inhibiting the degradation of peroxisomes increase the function of this organelle. It is possible that by simply increasing the half-life of peroxisomes, peroxisomal function can be improved by stabilizing the presence of peroxisomes. Peroxisomes can grow and divide as autonomous organelles, however, they can also be generated de novo (Dimitrov et al., 2013). There is evidence supporting the notion of the endoplasmic reticulum (ER) playing a role in the generation of new peroxisomal precursor vesicles that either mature on their own (Hoepfner et al., 2005), or fuse with one another to form a mature organelle (Lam et al., 2010). Since these precursor vesicles are thought to carry half a peroxisomal translocon complex, fusion initiates assembly of the full peroxisomal translocon, further allowing for the import of enzymes from the cytosol (van der Zand et al., 2012). Once the peroxisomes are able to import enzymes, they are thought to be mature. Thus, there is a population of “immature” and “mature” peroxisomes within the cell. Furthermore, the old peroxisomes are unable to fuse, as only ER-derived pre-peroxisomes (or the immature peroxisomes), are thought to be fusogenic (Dimitrov et al., 2013). Thus, it is possible that by inhibiting the degradation of peroxisomes, the pool of immature peroxisomes can be stabilized, (which may eventually fuse) and mature peroxisomes, resulting in fully functional peroxisomes.

In addition to bafilomycin, chloroquine and LY294002, it was found that two additional drugs that are FDA-approved also have the capability to restore peroxisome number and function (PTS1 import) in PEX1-mutated fibroblasts (data not shown). Difluoromethylornithine (DFMO, also known as eflornithine) and clomipramine have been used, which are currently used for the treatment of African trypanosomiasis (sleeping sickness) and depression, respectively. With the demonstration of various FDA-approved autophagy inhibitors being effective in improving peroxisome number and function, this enforces the possibility that these FDA-approved autophagy inhibitors could be re-purposed for the treatment of PBDs.

FDA-Approved Drug Chloroquine Recovers Peroxisome Number and Function without Compromising Cellular Viability.

Chloroquine was targeted, since it is a well-characterized autophagy inhibitor that is also currently FDA-approved for the treatment of malaria and rheumatoid arthritis. The strategy was to first determine an optimal concentration and incubation time whereby chloroquine improved peroxisome number and function, without compromising cellular viability.

The viability of control fibroblasts treated with chloroquine was examined by performing a MTT assay with 0, 1, 5, 10, 20, 50, and 100 uM chloroquine for 24-, 48-, 72- and 96-hours (FIG. 19, Panel A). Since the half-life of peroxisomes is 2-3 days, it was ideally sought to inhibit their degradation at this point. After 48-hours of incubation, treatment with 5 uM or 10 uM chloroquine resulted in 93% and 104% viability, respectively. Moreover, after 72-hours of incubation, treatment with 5 uM or 10 uM chloroquine resulted in 85% and 74% viability, respectively. Thus, with adequate cell viability, it was chosen to next examine peroxisome number and function with 5 and 10 uM chloroquine.

By using the PEX1-G843D-PTS1 cells, and treating these with either 5 uM or 10 uM chloroquine for 48- or 72-hours, a significant increase in GFP-PTS1 puncta was observed for both 5 uM and 10 uM at 24-, 48-, and 72-hours (FIG. 19, Panel B; FIG. 20, Panel C). An improvement in peroxisome number was also seen when PEX1-G843D-PTS1 cells were treated with 5 uM chloroquine for 48- and 72-hours, fixed and stained for PMP70 (FIG. 20, Panels D and E). This was further confirmed by probing whole cell lysates treated with 5 uM and 10 uM chloroquine for 48- and 72-hours for PMP70, Pex14, Catalase, and GAPDH (FIG. 21, Panel F). Probing for p62 and seeing an increase upon treatment with chloroquine also verifies that chloroquine is still able to inhibit autophagy at concentrations of 5 and 10 uM.

To investigate improvement in peroxisome function biochemically, VLCFA oxidation efficiency was examined using liquid chromatography-tandem mass spectrometry (LC-MS/MS), which has been previously described (Zhang et al., 2010). Peroxisomes are responsible for the β-oxidation of VLCFA, which are greater than 22-carbons in length. Treatment of PEX1-G843D homozygous or hemizygous primary fibroblasts with chloroquine for 144-hours (6 days) resulted in a significant reduction in C26:0 lysophosphatidylcholine levels in comparison to non-treated cells, which is indicative of an increase in β-oxidation metabolism (FIG. 21, Panel G). These results indicate that inhibiting autophagy may not only increase peroxisome numbers, but that indeed these peroxisomes may also function in a sense that the β-oxidation metabolism improves significantly.

Chloroquine May be a Long-Term Viable Treatment Option for PBD Patients.

The long-term viability of chloroquine treatment was further investigated by treating PEX1-G843D-PTS1 fibroblasts with 5 uM chloroquine for a 15-day span. On days 0, 3, 6, 9, 12, and 15, cells were harvested and their import capability (FIG. 22 and FIG. 23, Panel B) as well as their viability was assessed (FIG. 23, Panel C). A significant recovery in import was seen at all days with chloroquine treatment in comparison to the non-treated cells for each day. Furthermore, viability when treated with 5 uM chloroquine was either maintained (days 3, 9, 15) or improved (days 6, 12) in comparison to the non-treated cells. This indicates that chloroquine may be a viable long-term treatment option for patients with mild-intermediate PEX1 mutations.

### CONCLUSION

In summary, a model of AAA ATPase-deficient peroxisome degradation was characterized in a mammalian cell system, and it was shown that autophagy-inhibiting drugs may be a novel therapeutic approach to restore peroxisome number and function in PEX1-mutated PBD patients. Since there is currently no effective therapy for PBDs, identifying drugs and/or compounds to restore peroxisome number and function is imperative. Here, a model is characterized whereby stabilizing the half-life of peroxisomes (by inhibiting their degradation) can result in an increased pool of import-competent as well as 3-oxidation efficient peroxisomes. Based on the mechanism of damaged peroxisome degradation, as well as identification of drugs that can restore peroxisome number and function, clinical therapies for PBD patients are revealed.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

The following references are herein incorporated by reference to the extent permitted only in jurisdictions allowing incorporation of documentation by reference.
Apanasets, O., et al., (2014). PEX5, the shuttling import receptor for peroxisomal matrix proteins, is a redox-sensitive protein. Traffic Cph. Den. 15, 94-103.
Deosaran, E., et al. NBR1 acts as an autophagy receptor for peroxisomes. J Cell Sci 126, 939-952 (2013).
Dimitrov, L., Lam, S. K., and Schekman, R. (2013). The role of the endoplasmic reticulum in peroxisome biogenesis. Cold Spring Harb. Perspect. Biol. 5, a013243.
Fujiki et al., Biochim Biophys Acta (2012) June, 1822: 1337-1342.
Hoepfner, D., Schildknegt, D., Braakman, I., Philippsen, P., and Tabak, H. F. (2005). Contribution of the endoplasmic reticulum to peroxisome formation. Cell 122, 85-95.
Kim, P. K., Hailey, D. W., Mullen, R. T. & Lippincott-Schwartz, J. Ubiquitin signals autophagic degradation of cytosolic proteins and peroxisomes. Proc Natl Acad Sci USA 105, 20567-20574 (2008).
Ma, C., Schumann, U., Rayapuram, N. & Subramani, S. The peroxisomal matrix import of Pex8p requires only PTS receptors and Pex14p. Mol Biol Cell 20, 3680-3689 (2009).
Platta, H. W., Grunau, S., Rosenkranz, K., Girzalsky, W. & Erdmann, R. Functional role of the AAA peroxins in dislocation of the cycling PTS1 receptor back to the cytosol. Nat Cell Biol 7, 817-822 (2005).
Singh, I., et al. Impaired peroxisomal function in the central nervous system with inflammatory disease of experimental autoimmune encephalomyelitis animals and protection by lovastatin treatment. Brain Res 1022, 1-11 (2004).
Thorns, S. & Erdmann, R. Peroxisomal matrix protein receptor ubiquitination and recycling. Biochim Biophys Acta 1763, 1620-1628 (2006).
Uto, T., Contreras, M. A., Gilg, A. G. & Singh, I. Oxidative imbalance in nonstimulated X-adrenoleukodystrophy-derived lymphoblasts. Dev Neurosci 30, 410-418 (2008).
Van der Zand, A., Gent, J., Braakman, I., and Tabak, H. F. (2012). Biochemically Distinct Vesicles from the Endoplasmic Reticulum Fuse to Form Peroxisomes. Cell 149, 397-409.
Wei, H., Kemp, S., McGuinness, M. C., Moser, A. B. & Smith, K. D. Pharmacological induction of peroxisomes in peroxisome biogenesis disorders. Ann Neurol 47, 286-296 (2000).
Weller, S., Gould, S. J. & Valle, D. Peroxisome biogenesis disorders. Annu Rev Genomics Hum Genet 4, 165-211 (2003).
Yang, Y., Hu, L., Zheng, H., Mao, C., Hu, W., Xiong, K., Wang, F., and Liu, C. (2013). Application and interpretation of current autophagy inhibitors and activators. Acta Pharmacol. Sin. 34, 625-635.
Zhang, R., et al. Recovery of PEX1-Gly843Asp peroxisome dysfunction by small-molecule compounds. Proc Natl Acad Sci USA 107, 5569-5574 (2010).
Zheng et al., J Pharmacol Sci, (2014) 124:354-64.
International Patent Publication WO/1990/014094 A1
U.S. Pat. No. 4,151,281
U.S. Pat. No. 4,249,002
U.S. Pat. No. 4,309,442
U.S. Pat. No. 4,426,387
U.S. Pat. No. 5,707,608
U.S. Pat. No. 6,197,543
U.S. Pat. No. 7,994,222
U.S. Pat. No. 8,206,947
U.S. Pat. No. 8,343,996
U.S. Pat. No. 8,440,695
U.S. Pat. No. 8,466,311
U.S. Pat. No. 8,524,762.

1. A method of treating a peroxisome biogenesis disorder by administering to an individual in need thereof an effective amount of one or more autophagy inhibitor.

2. The method of claim 1 wherein the peroxisome biogenesis disorder comprises Zellweger syndrome, neonatal adrenoleukodystrophy, Infantile Refsum disease, or cerebrohepatorenal syndrome.

3. The method of claim 1 wherein the autophagy inhibitor comprises chloroquine diphosphate; hydroxychloroquine sulfate; verteporfin; difluoromethylornithine; clarithromycin; clomipramine; desmethylclomipramine hydrochloride, anisomycin; Spautin-1; U0126; SP600125; Wortmannin; LY294002; Bafilomycin A1; Forskolin; Melatonin; 1-((2-(diethylamino)ethyl)amino)-4-methylthioxanthen-9-one; 1-(2-diethylaminoethylamino)-4-(hydroxymethyl)-9-thioxanthenone; or N-[[1-[[2-(diethylamino)ethyl]amino]-9-oxo-9H-thiaxanthen-4-yl]methyl]methanesulfonamide.

4. The method of claim 1 wherein the autophagy inhibitor comprises chloroquine, LY294002, or Bafilomycin A1.

5. (canceled)

6. (canceled)

7. The method of claim 2 wherein the autophagy inhibitor comprises chloroquine diphosphate; hydroxychloroquine sulfate; verteporfin; difluoromethylornithine; clarithromycin; clomipramine; desmethylclomipramine hydrochloride, anisomycin; Spautin-1; U0126; SP600125; Wortmannin; LY294002; Bafilomycin A1; Forskolin; Melatonin; 1-((2-(diethylamino)ethyl)amino)-4-methylthioxanthen-9-one; 1-(2-diethylaminoethylamino)-4-(hydroxymethyl)-9-thioxanthenone; or N-[[1-[[2-(diethylamino)ethyl]amino]-9-oxo-9H-thiaxanthen-4-yl]methyl]methanesulfonamide.

8. The method of claim 2 wherein the autophagy inhibitor comprises chloroquine, LY294002, or Bafilomycin A1.

9. (canceled)

10. (canceled)

11. (canceled)

12. The method of claim 4 wherein the autophagy inhibitor comprises chloroquine.

13. A composition for use in treating a peroxisome biogenesis disorder in an individual in need thereof comprising one or more autophagy inhibitor and an acceptable diluent.

14. The composition of claim 13 wherein the peroxisome biogenesis disorder comprises Zellweger syndrome, neonatal adrenoleukodystrophy, Infantile Refsum disease, or cerebrohepatorenal syndrome.

15. The composition of claim 13 wherein the autophagy inhibitor comprises chloroquine diphosphate; hydroxychloroquine sulfate; verteporfin; difluoromethylornithine; clarithromycin; clomipramine; desmethylclomipramine hydrochloride, anisomycin; Spautin-1; U0126; SP600125; Wortmannin; LY294002; Bafilomycin A1; Forskolin; Melatonin; 1-((2-(diethylamino)ethyl)amino)-4-methylthioxanthen-9-one; 1-(2-diethylaminoethylamino)-4-(hydroxymethyl)-9-thioxanthenone; or N-[[1-[[2-(diethylamino)ethyl]amino]-9-oxo-9H-thiaxanthen-4-yl]methyl]methanesulfonamide.

16. The composition of claim 13 wherein the autophagy inhibitor comprises chloroquine, LY294002, or Bafilomycin A1.

17. A commercial package for treating a peroxisome biogenesis disorder comprising one or more autophagy inhibitor and instructions for use.

18. The commercial package of claim 17 wherein the peroxisome biogenesis disorder comprises Zellweger syndrome, neonatal adrenoleukodystrophy, Infantile Refsum disease, or cerebrohepatorenal syndrome.

19. The commercial package of claim 17 wherein the autophagy inhibitor comprises chloroquine diphosphate; hydroxychloroquine sulfate; verteporfin; difluoromethylornithine; clarithromycin; clomipramine; desmethylclomipramine hydrochloride, anisomycin; Spautin-1; U0126; SP600125; Wortmannin; LY294002; Bafilomycin A1; Forskolin; Melatonin; 1-((2-(diethylamino)ethyl)amino)-4-methylthioxanthen-9-one; 1-(2-diethylaminoethylamino)-4-(hydroxymethyl)-9-thioxanthenone; or N-[[1-[[2-(diethylamino)ethyl]amino]-9-oxo-9H-thiaxanthen-4-yl]methyl]methanesulfonamide.

20. The commercial package of claim 17 wherein the autophagy inhibitor comprises chloroquine, LY294002, or Bafilomycin A1.
